SEQUENCE LISTING (79 sequences)

SEQ ID NO: 1 - protein, 524 aa, Homo sapiens
MPTVEELYRNYGILADATEQVGQHKDAYQVILDGVKGGTKEKRLAAQFIPKFFKHFPELADSAINAQLDLCEDEDVSIRRQAIKELPQFATGENLPRVADILTQLLQTDDSAEFNLVNNALLSIFKMDAKGTLGGLFSQILQGEDIVRERAIKFLSTKLKTLPDEVLTKEVEELILTESKKVLEDVTGEEFVLFMKILSGLKSLQTVSGRQQLVELVAEQADLEQTFNPSDPDCVDRLLQCTRQAVPLFSKNVHSTRFVTYFCEQVLPNLGTLTTPVEGLDIQLEVLKLLAEMSSFCGDMEKLETNLRKLFDKLLEYMPLPPEEAENGENAGNEEPKLQFSYVECLLYSFHQLGRKLPDFLTAKLNAEKLKDFKIRLQYFARGLQVYIRQLRLALQGKTGEALKTEENKIKVVALKITNNINVLIKDLFHIPPSYKSTVTLSWKPVQKVEIGQKRASEDTTSGSPPKKSSAGPKRDARQIYNPPSGKYSSNLGNFNYEQRGAFRGSRGGRGWGTRGNRSRGRLY

SEQ ID NO: 2 - protein, 504 aa, Mus musculus
MPTVEELYRNYGILADATEQVGQHKDAYQVILDGVKGGTKEKRLAAQFIPKFFKHFPELADSAINAQLDLCEDEDVSIRRQAIKELPQFATGENLPRVADILTQLLQTDDSAEFNLVNNALLSIFKMDAKGTLGGLFSQILQGEDIVRERAIKFLSTKLKTLPDEVLTKEVEELILTESKKVLEDVTGEEFVLFMKILSGLKSLQTVSGRQQLVELVAEQADLEQAFSPSDPDCVDRLLQCTRQAVPLFSKNVHSTRFVTYFCEQVLPNLSTLTTPVEGLDIQLEVLKLLAEMSSFCGDMEKLETNLRKLFDKLLEYMPLPPEEAENGENAGNEEPKLQFSYVECLLYSFHQLGRKLPDFLTAKLNAEKLKDFKIRLQYFARGLQVYIRQLRLALQGKTGEALKTEENKIKVVALKITNNINVLIKDLFHIPPSYKSTVTLSWKPVQKVEIGQKRTSEDTSSGSPPKKSPGGPKRDARQIYNPPSGKYSSNLSNFNYERSLQGK

SEQ ID NO: 3 - protein, 481 aa, Artificial Sequence
  Description of Artificial Sequence Synthetic polypeptide
MKHHHHHHSSGLNDIFEAQKIEWHEENLYFQGSMPTVEELYRNYGILADATEQVGQHKDAYQVILDGVKGGTKEKRLAAQFIPKFFKHFPELADSAINAQLDLCEDEDVSIRRQAIKELPQFATGENLPRVADILTQLLQTDDSAEFNLVNNALLSIFKMDAKGTLGGLFSQILQGEDIVRERAIKFLSTKLKTLPDEVLTKEVEELILTESKKVLEDVTGEEFVLFMKILSGLKSLQTVSGRQQLVELVAEQADLEQTFNPSDPDCVDRLLQCTRQAVPLFSKNVHSTRFVTYFCEQVLPNLGTLTTPVEGLDIQLEVLKLLAEMSSFCGDMEKLETNLRKLFDKLLEYMPLPPEEAENGENAGNEEPKLQFSYVECLLYSFHQLGRKLPDFLTAKLNAEKLKDFKIRLQYFARGLQVYIRQLRLALQGKTGEALKTEENKIKVVALKITNNINVLIKDLFHIPPSYKSTVTLSWKPVQK

SEQ ID NO: 4 - protein, 481 aa, Artificial Sequence
  Description of Artificial Sequence Synthetic polypeptide
MKHHHHHHSSGLNDIFEAQKIEWHEENLYFQGSMPTVEELKRNKGILADATEQVGQHKDAYQVILDGVKGGTKEKRLAAQFIPKFFKHFPELADSAINAQLDLCEDEDVSIRRQAIKELPQFATGENLPRVADILTQLLQTDDSAEFNLVNNALLSIFKMDAKGTLGGLFSQILQGEDIVRERAIKFLSTKLKTLPDEVLTKEVEELILTESKKVLEDVTGEEFVLFMKILSGLKSLQTVSGRQQLVELVAEQADLEQTFNPSDPDCVDRLLQCTRQAVPLFSKNVHSTRFVTYFCEQVLPNLGTLTTPVEGLDIQLEVLKLLAEMSSFCGDMEKLETNLRKLFDKLLEYMPLPPEEAENGENAGNEEPKLQFSYVECLLYSFHQLGRKLPDFLTAKLNAEKLKDFKIRLQYFARGLQVYIRQLRLALQGKTGEALKTEENKIKVVALKITNNINVLIKDLFHIPPSYKSTVTLSWKPVQK

SEQ ID NO: 5 - protein, 481 aa, Artificial Sequence
  Description of Artificial Sequence Synthetic polypeptide
MKHHHHHHSSGLNDIFEAQKIEWHEENLYFQGSMPTVEELYRNYGILADATEQVGQHKDAYQVILDGVKGGTKEKRLAAQFIPKFFKHFPELADSAINAQLDLCEDEDVSIRRQAIKELPQFATGENLPRVADILTQLLQTDDSAEFNLVNNALLSIFKMDAKGTLGGLFSQILQGEDIVRERAIKFLSTKLKTLPDEVLTKEVEELILTESKKVLKKVTGEEFVLFMKILSGLKSLQTVSGRQQLVELVAEQADLEQTFNPSDPDCVDRLLQCTRQAVPLFSKNVHSTRFVTYFCEQVLPNLGTLTTPVEGLDIQLEVLKLLAEMSSFCGDMEKLETNLRKLFDKLLEYMPLPPEEAENGENAGNEEPKLQFSYVECLLYSFHQLGRKLPDFLTAKLNAEKLKDFKIRLQYFARGLQVYIRQLRLALQGKTGEALKTEENKIKVVALKITNNINVLIKDLFHIPPSYKSTVTLSWKPVQK

SEQ ID NO: 6 - protein, 239 aa, Artificial Sequence
  Description of Artificial Sequence Synthetic polypeptide
MKHHHHHHSSGLNDIFEAQKIEWHEENLYFQGSMPTVEELYRNYGILADATEQVGQHKDAYQVILDGVKGGTKEKRLAAQFIPKFFKHFPELADSAINAQLDLCEDEDVSIRRQAIKELPQFATGENLPRVADILTQLLQTDDSAEFNLVNNALLSIFKMDAKGTLGGLFSQILQGEDIVRERAIKFLSTKLKTLPDEVLTKEVEELILTESKKVLEDVTGEEFVLFMKILSGLKSLQT

SEQ ID NO: 7 - protein, 448 aa, Homo sapiens
MPTVEELYRNYGILADATEQVGQHKDAYQVILDGVKGGTKEKRLAAQFIPKFFKHFPELADSAINAQLDLCEDEDVSIRRQAIKELPQFATGENLPRVADILTQLLQTDDSAEFNLVNNALLSIFKMDAKGTLGGLFSQILQGEDIVRERAIKFLSTKLKTLPDEVLTKEVEELILTESKKVLEDVTGEEFVLFMKILSGLKSLQTVSGRQQLVELVAEQADLEQTFNPSDPDCVDRLLQCTRQAVPLFSKNVHSTRFVTYFCEQVLPNLGTLTTPVEGLDIQLEVLKLLAEMSSFCGDMEKLETNLRKLFDKLLEYMPLPPEEAENGENAGNEEPKLQFSYVECLLYSFHQLGRKLPDFLTAKLNAEKLKDFKIRLQYFARGLQVYIRQLRLALQGKTGEALKTEENKIKVVALKITNNINVLIKDLFHIPPSYKSTVTLSWKPVQK

SEQ ID NO: 8 - protein, 206 aa, Homo sapiens
MPTVEELYRNYGILADATEQVGQHKDAYQVILDGVKGGTKEKRLAAQFIPKFFKHFPELADSAINAQLDLCEDEDVSIRRQAIKELPQFATGENLPRVADILTQLLQTDDSAEFNLVNNALLSIFKMDAKGTLGGLFSQILQGEDIVRERAIKFLSTKLKTLPDEVLTKEVEELILTESKKVLEDVTGEEFVLFMKILSGLKSLQT

SEQ ID NO: 9 - protein, 25 aa, Artificial Sequence
  Description of Artificial Sequence Synthetic peptide
MKHHHHHHSSGLNDIFEAQKIEWHE

SEQ ID NO: 10 - protein, 8 aa, Artificial Sequence
  Description of Artificial Sequence Synthetic peptide
ENLYFQGS

SEQ ID NO: 11 - protein, 481 aa, Artificial Sequence
  Description of Artificial Sequence Synthetic polypeptide
MKHHHHHHSSGLNDIFEAQKIEWHEENLYFQGSMPTVEELKRNKGILADATEQVGQHKDAYQVILDGVKGGTKEKRLAAQFIPKFFKHFPELADSAINAQLDLCEDEDVSIRRQAIKELPQFATGENLPRVADILTQLLQTDDSAEFNLVNNALLSIFKMDAKGTLGGLFSQILQGEDIVRERAIKFLSTKLKTLPDEVLTKEVEELILTESKKVLKKVTGEEFVLFMKILSGLKSLQTVSGRQQLVELVAEQADLEQTFNPSDPDCVDRLLQCTRQAVPLFSKNVHSTRFVTYFCEQVLPNLGTLTTPVEGLDIQLEVLKLLAEMSSFCGDMEKLETNLRKLFDKLLEYMPLPPEEAENGENAGNEEPKLQFSYVECLLYSFHQLGRKLPDFLTAKLNAEKLKDFKIRLQYFARGLQVYIRQLRLALQGKTGEALKTEENKIKVVALKITNNINVLIKDLFHIPPSYKSTVTLSWKPVQK

SEQ ID NO: 12 - protein, 11 aa, Artificial Sequence
  Description of Artificial Sequence Synthetic peptide
DICLPRWGCLW

SEQ ID NO: 13 - protein, 18 aa, Artificial Sequence
  Description of Artificial Sequence Synthetic peptide
RLIEDICLPRWGCLWEDD

SEQ ID NO: 14 - protein, 20 aa, Artificial Sequence
  Description of Artificial Sequence Synthetic peptide
QRLMEDICLPRWGCLWEDDF

SEQ ID NO: 15 - protein, 21 aa, Artificial Sequence
  Description of Artificial Sequence Synthetic peptide
QGLIGDICLPRWGCLWGDSVK

SEQ ID NO: 16 - protein, 20 aa, Artificial Sequence
  Description of Artificial Sequence Synthetic peptide
GEWWEDICLPRWGCLWEEED

SEQ ID NO: 17 - protein, 20 aa, Artificial Sequence
  Description of Artificial Sequence Synthetic peptide
QRLIEDICLPRWGCLWEDDF

SEQ ID NO: 18 - protein, 17 aa, Artificial Sequence
  Description of Artificial Sequence Synthetic peptide
RLIEDICLPRWGCLWED

SEQ ID NO: 19 - protein, 16 aa, Artificial Sequence
  Description of Artificial Sequence Synthetic peptide
RLIEDICLPRWGCLWE

SEQ ID NO: 20 - protein, 15 aa, Artificial Sequence
  Description of Artificial Sequence Synthetic peptide
RLIEDICLPRWGCLW

SEQ ID NO: 21 - protein, 16 aa, Artificial Sequence
  Description of Artificial Sequence Synthetic peptide
LIEDICLPRWGCLWED

SEQ ID NO: 22 - protein, 20 aa, Artificial Sequence
  Description of Artificial Sequence Synthetic peptide
EVRSFCTDWPAEKSCKPLRG

SEQ ID NO: 23 - protein, 20 aa, Artificial Sequence
  Description of Artificial Sequence Synthetic peptide
RAPESFVCYWETICFERSEQ

SEQ ID NO: 24 - protein, 11 aa, Artificial Sequence
  Description of Artificial Sequence Synthetic peptide
EMCYFPGICWM

SEQ ID NO: 25 - protein, 20 aa, Artificial Sequence
  Description of Artificial Sequence Synthetic peptide
GENWCDSTLMAYDLCGQVNM

SEQ ID NO: 26 - protein, 20 aa, Artificial Sequence
  Description of Artificial Sequence Synthetic peptide
MDELAFYCGIWECLMHQEQK

SEQ ID NO: 27 - protein, 10 aa, Artificial Sequence
  Description of Artificial Sequence Synthetic peptide
DLCDVDFCWF

SEQ ID NO: 28 - protein, 16 aa, Artificial Sequence
  Description of Artificial Sequence Synthetic peptide
KSCSELHWLLVEECLF

SEQ ID NO: 29 - protein, 20 aa, Artificial Sequence
  Description of Artificial Sequence Synthetic peptide
RNEDPCVVLLEMGLECWEGV

SEQ ID NO: 30 - protein, 15 aa, Artificial Sequence
  Description of Artificial Sequence Synthetic peptide
DTCVDLVRLGLECWG

SEQ ID NO: 31 - protein, 20 aa, Artificial Sequence
  Description of Artificial Sequence Synthetic peptide
QRQMVDFCLPQWGCLWGDGF

SEQ ID NO: 32 - protein, 11 aa, Artificial Sequence
  Description of Artificial Sequence Synthetic peptide
DLCLRDWGCLW

SEQ ID NO: 33 - protein, 20 aa, Artificial Sequence
  Description of Artificial Sequence Synthetic peptide
QRQMVDFCLPQWGCLWGDGF

SEQ ID NO: 34 - protein, 20 aa, Artificial Sequence
  Description of Artificial Sequence Synthetic peptide
QRHPEDICLPRWGCLWGDDD

SEQ ID NO: 35 - protein, 20 aa, Artificial Sequence
  Description of Artificial Sequence Synthetic peptide
NRQMEDICLPQWGCLWGDDF

SEQ ID NO: 36 - protein, 20 aa, Artificial Sequence
  Description of Artificial Sequence Synthetic peptide
QRLMEDICLPRWGCLWGDRF

SEQ ID NO: 37 - protein, 20 aa, Artificial Sequence
  Description of Artificial Sequence Synthetic peptide
QWHMEDICLPQWGCLWGDVL

SEQ ID NO: 38 - protein, 20 aa, Artificial Sequence
  Description of Artificial Sequence Synthetic peptide
QWQMENVCLPKWGCLWEELD

SEQ ID NO: 39 - protein, 20 aa, Artificial Sequence
  Description of Artificial Sequence Synthetic peptide
LWAMEDICLPKWGCLWEDDF

SEQ ID NO: 40 - protein, 20 aa, Artificial Sequence
  Description of Artificial Sequence Synthetic peptide
LRLMDNICLPRWGCLWDDGF

SEQ ID NO: 41 - protein, 20 aa, Artificial Sequence
  Description of Artificial Sequence Synthetic peptide
HSQMEDICLPRWGCLWGDEL

SEQ ID NO: 42 - protein, 20 aa, Artificial Sequence
  Description of Artificial Sequence Synthetic peptide
QWQVMDICLPRWGCLWADEY

SEQ ID NO: 43 - protein, 20 aa, Artificial Sequence
  Description of Artificial Sequence Synthetic peptide
QGLIGDICLPRWGCLWGDSV

SEQ ID NO: 44 - protein, 20 aa, Artificial Sequence
  Description of Artificial Sequence Synthetic peptide
HRLVEDICLPRWGCLWGNDF

SEQ ID NO: 45 - protein, 20 aa, Artificial Sequence
  Description of Artificial Sequence Synthetic peptide
QMHMMDICLPKWGCLWGDTS

SEQ ID NO: 46 - protein, 20 aa, Artificial Sequence
  Description of Artificial Sequence Synthetic peptide
LRIFEDICLPKWGCLWGEGF

SEQ ID NO: 47 - protein, 20 aa, Artificial Sequence
  Description of Artificial Sequence Synthetic peptide
QSYMEDICLPRWGCLSDDAS

SEQ ID NO: 48 - protein, 20 aa, Artificial Sequence
  Description of Artificial Sequence Synthetic peptide
QGDFWDICLPRWGCLSGEGY

SEQ ID NO: 49 - protein, 20 aa, Artificial Sequence
  Description of Artificial Sequence Synthetic peptide
RWQTEDVCLPKWGCLFGDGV

SEQ ID NO: 50 - protein, 20 aa, Artificial Sequence
  Description of Artificial Sequence Synthetic peptide
QGLIGDICLPRWGCLWGDSV

SEQ ID NO: 51 - protein, 20 aa, Artificial Sequence
  Description of Artificial Sequence Synthetic peptide
LIFMEDVCLPQWGCLWEDGV

SEQ ID NO: 52 - protein, 20 aa, Artificial Sequence
  Description of Artificial Sequence Synthetic peptide
QRDMGDICLPRWGCLWEDGV

SEQ ID NO: 53 - protein, 20 aa, Artificial Sequence
  Description of Artificial Sequence Synthetic peptide
QRHMMDFCLPKWGCLWGDGY

SEQ ID NO: 54 - protein, 20 aa, Artificial Sequence
  Description of Artificial Sequence Synthetic peptide
QRPIMDFCLPKWGCLWEDGF

SEQ ID NO: 55 - protein, 20 aa, Artificial Sequence
  Description of Artificial Sequence Synthetic peptide
ERQMVDFCLPKWGCLWGDGF

SEQ ID NO: 56 - protein, 20 aa, Artificial Sequence
  Description of Artificial Sequence Synthetic peptide
QGYMVDFCLPRWGCLWGDAN

SEQ ID NO: 57 - protein, 20 aa, Artificial Sequence
  Description of Artificial Sequence Synthetic peptide
KMGRVDFCLPKWGCLWGDEL

SEQ ID NO: 58 - protein, 20 aa, Artificial Sequence
  Description of Artificial Sequence Synthetic peptide
QSQLEDFCLPKWGCLWGDGF

SEQ ID NO: 59 - protein, 20 aa, Artificial Sequence
  Description of Artificial Sequence Synthetic peptide
QGGMGDFCLPQWGCLWGEDL

SEQ ID NO: 60 - protein, 20 aa, Artificial Sequence
  Description of Artificial Sequence Synthetic peptide
QRLMWEICLPLWGCLWGDGL

SEQ ID NO: 61 - protein, 20 aa, Artificial Sequence
  Description of Artificial Sequence Synthetic peptide
QRQIMDFCLPHWGCLWGDGF

SEQ ID NO: 62 - protein, 20 aa, Artificial Sequence
  Description of Artificial Sequence Synthetic peptide
GRQVVDFCLPKWGCLWEEGL

SEQ ID NO: 63 - protein, 20 aa, Artificial Sequence
  Description of Artificial Sequence Synthetic peptide
QMQMSDFCLPQWGCLWGDGY

SEQ ID NO: 64 - protein, 20 aa, Artificial Sequence
  Description of Artificial Sequence Synthetic peptide
KSRMGDFCLPEWGCLWGDEL

SEQ ID NO: 65 - protein, 20 aa, Artificial Sequence
  Description of Artificial Sequence Synthetic peptide
ERQMEDFCLPQWGCLWGDGV

SEQ ID NO: 66 - protein, 20 aa, Artificial Sequence
  Description of Artificial Sequence Synthetic peptide
QRQVVDFCLPQWGCLWGDGS

SEQ ID NO: 67 - protein, 18 aa, Artificial Sequence
  Description of Artificial Sequence Synthetic peptide
WWEQDRDWDFDVFGGGTP

SEQ ID NO: 68 - protein, 7 aa, Artificial Sequence
  Description of Artificial Sequence Synthetic peptide
EYEKEYE

SEQ ID NO: 69 - protein, 18 aa, Artificial Sequence
  Description of Artificial Sequence Synthetic peptide
RLIEDICLPRWGCLWEDD

SEQ ID NO: 70 - protein, 10 aa, Artificial Sequence
  Description of Artificial Sequence Synthetic peptide
  MOD_RES (2)..(3): Variable lysine
  MOD_RES (6)..(6): Variable lysine
  MOD_RES (8)..(8): Variable lysine
AKKPGKAKPG

SEQ ID NO: 71 - protein, 232 aa, Homo sapiens
EPKSCDKTHTCPPCPAPELLGGPSVFLEPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 232 aa, Artificial Sequence
  Description of Artificial Sequence Synthetic polypeptide
EPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 73 - protein, 12 aa, Artificial Sequence
  Description of Artificial Sequence Synthetic peptide
GSGEGEGSEGSG

SEQ ID NO: 74 - protein, 12 aa, Artificial Sequence
  Description of Artificial Sequence Synthetic peptide
GGSEGEGSEGGS

SEQ ID NO: 75 - protein, 5 aa, Artificial Sequence
  Description of Artificial Sequence Synthetic peptide
GGGGS

SEQ ID NO: 76 - protein, 107 aa, Artificial Sequence
  Description of Artificial Sequence Synthetic polypeptide
MGVSDVPRDLEVVAATPTSLLISWDAPAVTVRYYRITYGREVQKYSDLGPLYIYQEFTVPGSKSTATISGLKPGVDYTITVYAVTGSGESPASSKPISINYRTEIDK

SEQ ID NO: 77 - protein, 107 aa, Artificial Sequence
  Description of Artificial Sequence Synthetic polypeptide
MGVSDVPRDLEVVAATPTSLLISWDAPAVTVRYYRITYGREVQKYSDWGPLYIYNEFTVPGSKSTATISGLKPGVDYTITVYAVTGSGESPASSKPISINYRTEIDK

SEQ ID NO: 78 - protein, 6 aa, Artificial Sequence
  Description of Artificial Sequence Synthetic 6xHis tag
HHHHHH

SEQ ID NO: 79 - protein, 4 aa, Artificial Sequence
  Description of Artificial Sequence Synthetic peptide
GGGS

(19) United States
(12) Patent Application Publication
(10) Pub. No.: US 2024/0317829 A1
(43) Pub. Date: Sep. 26, 2024

(54) APPLICATION OF APOPTOSIS INHIBITOR 5 (API5) FOR EPITHELIAL RESTITUTION

(71) Applicant: New York University, NY, NY (US)

(72) Inventors: Kenneth Cadwell, NY, NY (US); Shohei Koide, NY, NY (US); Yu Matsuzawa-Ishimoto, NY, NY (US); Akiko Koiide, NY, NY (US)

(73) Assignee: New York University, NY, NY (US)

(21) Appl. No.: 18/280,186

(22) PCT Filed: Mar. 7, 2022
(86) PCT No.: PCT/US2022/019138

§ 371 Date: Sep. 1, 2023

Related U.S. Application Data
(60) Provisional application No. 63/157,225, filed on Mar. 5, 2021.

(51) Int. Cl. C07K 14/47; A61K 38/17; A61K 45/06; A61P 1/04

(52) U.S. Cl. CPC C07K 14/4747; A61K 38/1761; A61K 45/06; A61P 1/04; C07K 2319/21; C07K 2319/50

(57) ABSTRACT

The present disclosure provides, among other things, recombinant API5 proteins and isolated nucleic acids encoding the same. Also provided are vectors comprising the nucleic acids, and host cells comprising the vectors or nucleic acids encoding the recombinant API5 proteins. Further provided are compositions comprising such recombinant proteins, and the methods of using these recombinant proteins for epithelial restitution and treatment of related diseases and disorders.

### CROSS-REFERENCE TO RELATED APPLICATIONS

This patent application claims priority to U.S. Provisional Application No. 63/157,225, filed Mar. 5, 2021, the disclosure of each of which is herein incorporated by reference in its entirety.

### STATEMENT REGARDING FEDERALLY SPONSORED RESEARCH

This invention was made with government support under DK093668 awarded by National Institutes of Health. The government has certain rights in the invention.

### FIELD OF THE INVENTION

The present invention relates to recombinant apoptosis inhibitor 5 (API5) proteins. The invention further relates to compositions comprising such recombinant proteins, and the use of these recombinant proteins for epithelial restitution and treatment of related diseases and disorders.

### SEQUENCE LISTING

The instant application contains a Sequence Listing which has been submitted electronically in ASCII format and is hereby incorporated by reference in its entirety. Said ASCII copy, created on Mar. 2, 2022, is named 243735_000245_SL.txt and is 60,412 bytes in size.

### BACKGROUND

Immune-mediated damage to the epithelial barrier is considered the central event in the pathogenesis of inflammatory bowel diseases (IBDs) such as Crohn's disease. Although numerous strategies targeting immune effectors have been developed including those blocking TNFα or lymphocyte migration, these interventions do not discriminate between pathologic inflammation and immune processes necessary to maintain homeostasis with the gut microbiota. Strategies that enhance the resilience of the epithelium to immune-mediated injury may be effective in promoting long-term remission without compromising the immune system. However, no such therapies currently exist. This present application addresses this and other related needs.

### SUMMARY OF THE INVENTION

In one aspect, provided herein is a recombinant protein comprising an apoptosis inhibitor 5 (API5) protein, or a fragment or a variant thereof. In some embodiments, the API5 protein comprises the amino acid sequence of SEQ ID NO: 1 or 2, or a sequence having at least 90% identity thereto. In some embodiments, the fragment of API5 comprises the N-terminal HEAT repeat region of API5. In some embodiments, the fragment of API5 comprises residues 1-448 of SEQ ID NO: 1. In some embodiments, the fragment of API5 comprises residues 1-206 of SEQ ID NO: 1.

In some embodiments, the API5 protein, or a fragment or a variant thereof, is genetically fused to and/or chemically conjugated to one or more heterologous moieties. In some embodiments, the one or more heterologous moieties comprise one or more affinity tags. In some embodiments, the affinity tag is a His tag, an Avi-tag, a hemagglutinin (HA) tag, a FLAG tag, a Myc tag, a GST tag, a MBP tag, a chitin binding protein tag, a calmodulin tag, a V5 tag, a streptavidin binding tag, a green fluorescent protein (GFP), YFP, RFP, CFP, mCherry, tdTomato, SUMO tag, Ubiquitin tag, or a combination thereof.

In some embodiments, the one or more heterologous moieties comprise a His₆ tag (SEQ ID NO: 78) and an Avi-tag and optionally comprise the amino acid sequence of MKHHHHHHSSGLNDIFEAQKIEWHE (SEQ ID NO: 9). In some embodiments, the recombinant protein further comprises a protease cleavage site between the API5 protein, or a fragment or a variant thereof, and the one or more affinity tags. In some embodiments, the protease cleavage site is a cleavage site for the TEV protease and optionally comprises the amino acid sequence of ENLYFQGS (SEQ ID NO: 10).

In one embodiment, the recombinant protein comprises the amino acid sequence of SEQ ID NO: 3.

In one embodiment, the recombinant protein comprises the amino acid sequence of SEQ ID NO: 6.

In some embodiments, the one or more heterologous moieties comprise a moiety that specifically binds albumin. In some embodiments, the moiety that specifically binds albumin comprises the amino acid sequence of any one of SEQ ID NOs: 12-66, 76 and 77. In some embodiments, the moiety that specifically binds albumin is selected from Naphthalene acylsulfonamide, Diphenylcyclohexanol phosphate ester, 9-fluorenylmethoxycarbonyl (Fmoc), Fmoc derivative linked to a 16-sulfanylhexadecanoic acid through a maleimide group, Dicoumarol derivative with maleimide, Evans blue derivative with maleimide, Diflunisal-γGlu-Lys(±O2O c)-indomethacin, lithocholic acid coupled to a γGlu linker, 6-(4-(p-Iodophenyl) butanamido) hexanoate, A083/B134, A099/B344, 89D03 (Ac-WWEQDRDWDFDVFGGGTP-NH2, SEQ ID NO: 67), acylated heptapeptide F-tag (fluorescein-EYEK(palmitate)EYE-NH2, SEQ ID NO: 68), disulfide cyclized peptide SA21 (Ac-RLIEDICLPRWGCLWEDD-NH₂, SEQ ID NO: 69), head-to-tail cyclized peptide HSA-1 (AK*K*PGK*AK*PG with variable lysine (K*), SEQ ID NO: 70), ABD035, ABDCon, DARPins, AlbudAbs, dsFv CA645, Nanobody Nb.b201, and VNAR E06. In some embodiments, the albumin is rat albumin, rabbit albumin, or human albumin. In some embodiments, the moiety that specifically binds albumin is genetically fused or chemically conjugated to the N-terminus of the API5 protein, or a fragment or a variant thereof. In some embodiments, the moiety that specifically binds albumin is genetically fused or chemically conjugated to the C-terminus of the API5 protein, or a fragment or a variant thereof. In some embodiments, the moiety that specifically binds albumin is genetically fused or chemically conjugated to the API5 protein, or a fragment or a variant thereof via a linker (e.g., peptidyl linker or nonpeptidyl linker).

In some embodiments, the one or more heterologous moieties comprise a human IgG Fc domain. In some embodiments, the Fc domain is modified to alter effector function of the domain. In some embodiments, the Fc domain is modified to enhance the half-life of the recombinant protein.

In some embodiments, the one or more heterologous moieties comprise an albumin.

In some embodiments, the one or more heterologous moieties comprise a polyethylene glycol (PEG) polymer. In some embodiments, the recombinant protein is modified to introduce one or more glycosylation sites in the recombinant protein.

In another aspect, provided herein is an isolated polynucleotide encoding the recombinant protein described herein. In some embodiments, the isolated polynucleotide is an mRNA.

In another aspect, provided herein is a vector comprising the polynucleotide described herein.

In another aspect, provided herein is a host cell comprising the polynucleotide or the vector described herein.

In another aspect, provided herein is a pharmaceutical composition comprising the recombinant protein, the polynucleotide, or the vector described herein, and a pharmaceutically acceptable carrier or excipient.

In another aspect, provided herein is a method of producing a recombinant API5 protein described herein, comprising growing the host cell described herein under conditions where the API5 protein encoded by the polynucleotide is expressed. The method may further comprise isolating the protein.

In another aspect, provided herein is a method of protecting an epithelial cell from cell death, comprising contacting the epithelial cell with a therapeutically effective amount of the recombinant protein, the polynucleotide, or the vector described herein, or a pharmaceutical composition thereof. In some embodiments, the epithelial cell is an intestinal epithelial cell. In some embodiments, the epithelial cell is a Paneth cell.

In another aspect, provided herein is a method of restoring an intestinal epithelial barrier in a subject in need thereof, comprising administering to the subject a therapeutically effective amount of the recombinant protein, the polynucleotide, the vector described herein, or a pharmaceutical composition thereof. In some embodiments, the subject has a gastrointestinal disease. In some embodiments, the gastrointestinal disease is an inflammatory bowel disease, graft-versus-host disease, pouchitis, immune checkpoint inhibitor associated colitis, radiation induced gastrointestinal toxicity, irritable bowel syndrome, short bowel syndrome, infectious gastroenteritis, or celiac disease. In some embodiments, the inflammatory bowel disease is Crohn's disease. In some embodiments, the inflammatory bowel disease is ulcerative colitis.

In another aspect, provided herein is a method of treating a gastrointestinal disease in a subject in need thereof, comprising administering to the subject a therapeutically effective amount of the recombinant protein, the polynucleotide, or the vector described herein, or a pharmaceutical composition thereof. In some embodiments, the gastrointestinal disease is inflammatory bowel disease, graft-versus-host disease, pouchitis, immune checkpoint inhibitor associated colitis, radiation induced gastrointestinal toxicity, irritable bowel syndrome, short bowel syndrome, infectious gastroenteritis, or celiac disease. In some embodiments, the inflammatory bowel disease is Crohn's disease. In some embodiments, the inflammatory bowel disease is ulcerative colitis.

In various embodiments of the methods described herein, the recombinant protein, the polynucleotide, the vector, or the pharmaceutical composition is administered intravenously, orally, intrarectally, or via delivery through endoscopy.

In some embodiments, the method described herein further comprises administering one or more additional agents. The one or more additional agents may inhibit TNFα and/or lymphocyte migration. In some embodiments, the one or more additional agent comprise an integrin inhibitor or a sphingosine-1-phosphate (SIP) receptor modulator. In some embodiments, the integrin inhibitor is vedolizumab (Entyvio), etrolizumab, PN-943, ZP10000, or MORF-057. In some embodiments, the SIP receptor modulator is fingolimod, ozanimod, etrasimod, or amiselimod.

In various embodiments of the methods described herein, the subject is human.

### BRIEF DESCRIPTION OF THE DRAWINGS

FIGS. 1A-1C show that γδ T cells protect Paneth cells and intestinal organoids from cell death. FIGS. 1A-1B show that addition of intra-epithelial lymphocytes (IELs) to Atg16L1^{-/-} organoids restores viability (FIG. 1A) and the proportion (FIG. 1B) of Paneth cells to similar levels as control Atg16L1^{+/+} wild-type organoids. FIG. 1C shows that among the different IEL subtypes, γδ T cells were the ones that mediate protection of Atg16L1^{-/-} organoids. Each dot in FIG. 1A and FIG. 1C represents an independent biological repeat from different mice. Each dot in FIG. 1B represents a field under the microscope. *p<0.01, ****p<0.0001.

FIGS. 2A-2D show inhibition of the protective function of γδ T cells is associated with Paneth cell defects. FIG. 2A shows that murine norovirus (MNV) inhibits γδ T cell mobility, a sign that their activity is altered. FIG. 2B shows that γδ T cells from uninfected mice, but not MNV-infected mice, promote Atg16L1^{-/-} organoid viability, indicating the virus interferes with the protective effect of these cells. FIG. 2C shows that double knockout mice generated by crossing Atg16L1^{-/-} mice with mice that lack γδ T cells (Tcrd^{-/-}) display a loss of Paneth cells. FIG. 2D shows the results from lysozyme immunofluorescence microscopy, which indicated that the remaining Paneth cells displayed abnormal staining patterns of this antimicrobial molecule in Atg16L1^{-/-} Tcrd^{-/-} mice compared to single knockout controls. Dots represent individual cells in FIG. 2A, independent repeats from different mice in FIG. 2B, and individual mice in FIG. 2C and FIG. 2D. ****p<0.0001.

FIG. 3 depicts a Venn diagram showing the number of overlapping and distinct proteins in the supernatant samples from FACS-sorted TCRγδ⁺ cells and TCRαβ⁺ cells.

FIGS. 4A-4F show that API5 protects intestinal organoids and restores Paneth cells. FIG. 4A shows that 50 nM recombinant human API5 (rhAPI5) restores Atg16L1^{-/-} organoid viability. FIG. 4B displays hematoxylin and eosin (H&E)-stained sections showing that rhAPI5 restores Paneth cells (arrows). Scale bar=50 m. FIGS. 4C-4D show quantification of absolute Paneth cell numbers per organoid (FIG. 4C) and percent of total intestinal epithelial cells (IECs) (FIG. 4D), confirming a restoration of Paneth cells. FIG. 4E shows that total IECs do not increase indicating that the effect of rAPI5 is Paneth cell-specific and not as a non-specific growth factor. FIG. 4F shows that adding IEL supernatant in which API5 is depleted with an antibody exacerbates Atg16L1^{-/-} organoid death, while adding rAPI5 to the depleted supernatant results in similar protection as the intact IEL supernatant (Control sup). N=3 mice/condition in 3 independent repeats. **p<0.01, ***p<0.001, ****p<0.0001.

FIG. 5 shows that binding interface residues of API5 are necessary for protective effects. First two bar graphs are controls showing that 50 nM recombinant human API5 (rhAPI5) restores Atg16L1^{-/-} organoid viability as previously indicated. API5 mutant 1 (Y8K;Y11K), mutant 2 (E184K;D185K), and mutant 3 (Y8K;Y11K; E184K;D185K) abrogated the protective activity, even when adding excess protein up to 500 nM. N=3 mice/condition in 3 independent repeats. ****p<0.0001.

FIG. 6 shows that API5 prevents TNFα-induced loss of epithelial viability. Atg16L1^{-/-} organoids undergo exacerbated necrotic cell death in the presence of 20 ng/ml TNFα due to their loss of Paneth cells, but control organoids are resistant. Administering 50 nM rhAPI5 prevents the toxic effect of TNFα to improve the viability of Atg16L1^{-/-} organoids. Left panel shows quantification of 3 independent repeats and right panels show representative pictures of Atg16L1^{-/-} organoids on day 5 post-differentiation following 48 hrs of the indicated treatments. ***p<0.001, ****p<0.0001.

FIG. 7 shows the sequence alignment of human and mouse API5 proteins. FIG. 7 discloses SEQ ID NOS 1-2, respectively, in order of appearance.

FIGS. 8A-8F demonstrate that API5 prevents Paneth cell loss and protects against intestinal injury in Atg16L1 mutant mice. FIG. 8A shows that administration of API5 restores Paneth cells and reduces cell death in mice deficient in both Atg16L1 and γδ T cells. As shown in FIG. 2C, mice deficient in both Atg16L1 and γδ T cells (Atg16L1^{ΔIEC} TCRδ^{-/-}) display a reduction in Paneth cells compared with mice that have ATG16L1 intact (Atg16L1^{f/f} TCRδ^{-/-}). Intravenously injecting Atg16L1^{ΔIEC} TCRδ^{-/-} mice (abbreviated as ΔIEC TCRδ^{-/-} in FIG. 8A) with 40 μg wild-type recombinant human API5 (rAPI5^{WT}) but not the control variant protein rAPI5^{Y8K:Y11K} reversed this defect according to quantification of Paneth cells in H&E-stained sections and dead Paneth cells in terminal deoxynucleotidyl transferase dUTP nick end labeling (TUNEL)-stained sections. Atg16L1^{f/f} TCRδ^{-/-} mice (abbreviated as f/f TCRδ^{-/-}) is shown as a reference. n=7 (f/f TCRδ^{-/-} rAPI5^{Y8K:Y11K}), 8 (ΔIEC TCRδ^{-/-} rAPI5^{Y8K:Y11K}), 8 (f/f TCRδ^{-/-} rAPI5^{WT}), 10 (ΔIEC TCRδ^{-/-} rAPI5^{WT}). FIG. 8B shows Western blot analysis demonstrating reduced secretion of API5 by γδ T cells in mice with partial API5 deficiency. CRISPR-Cas9 was used to delete Api5. Heterozygous knockout mice (Api5^{+/-}) were used because homozygous knockout mice displayed insufficient viability for experiments. Western blot analysis of γδ supernatant (SN) and cell lysate harvested from Api5^{+/+} or Api5^{+/-} mice shows that heterozygosity leads to a reduction in API5 secretion. PGRP-L is the loading control for the supernatant. FIGS. 8C-8D show representative images and quantification of H&E (FIG. 8C) and lysozyme staining (FIG. 8D) of small intestinal tissue from Atg16L1^{f/f} Api5^{+/-} (f/f Api5^{+/-}) and Atg16L1^{ΔIEC} Api5^{+/-} (ΔIEC Api5^{+/-}) mice. The results show that heterozygous knockout of Api5 leads to reduction in Paneth cells with intact granules (arrowheads). n=6 (f/f Api5^{+/-}) and 5 (ΔIEC Api5^{+/-}). Scale bar 20 μm. FIGS. 8E-8F show that Atg16L1^{ΔPC} (ΔPC) mice in which Atg16L1 is deleted from Paneth cells (defensin-Cre Atg16L1^{f/f}) display reduced survival (FIG. 8E) and higher disease scores (FIG. 8F) compared with their littermate controls (f/f) following chemical injury to the gut with 5% DSS for 6 days. Therapeutic intravenous administration of 40 μg/mouse of rAPI5^{WT} protein on day 0, 3, and 6 led to 100% survival of ΔPC mice along with significant improvement in signs of disease, while control protein rAPI5^{Y8K:Y11K} had no effect. Treatment of f/f mice did not have an effect. n=7 (f/f), 8 (ΔPC), n=7 (f/f rAPI5^{WT}), 9 (ΔPC rAPI5^{WT}), n=6 (f/f rAPI5^{Y8K:Y11K}), 7 (ΔPC rAPI5^{Y8K:Y11K}). Data points bar graphs represent individual mice. Bars represent means±SEM, and survival data in FIG. 8E are combined results of 2 experiments performed independently. ***p<0.001, ****p<0.0001.

### DETAILED DESCRIPTION

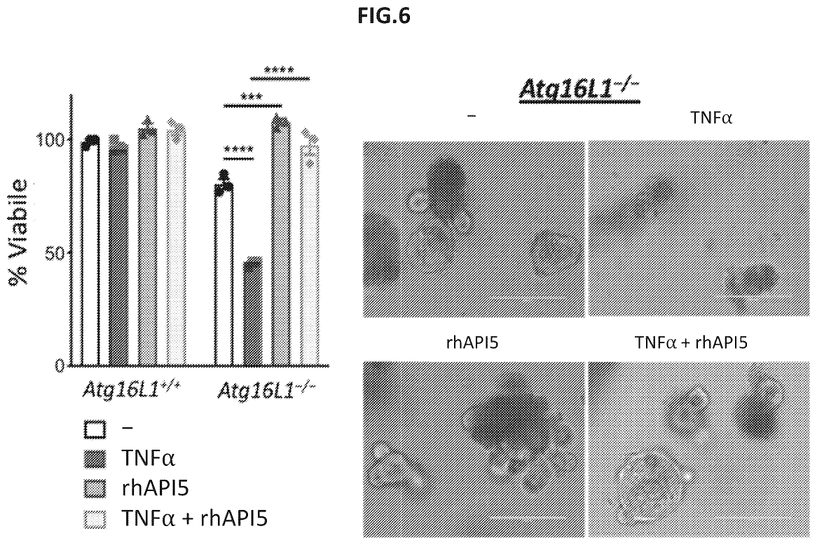

The present application is based, in part, on the surprising and unexpected discovery that a novel factor, apoptosis inhibitor 5 (API5), can protect intestinal epithelial cells (IECs), especially Paneth cells, from immune-mediated damage. API5 was found to ameliorate inflammatory disease of the gastrointestinal tract by inhibiting cytokine-mediated damage to the epithelium. As detailed in the Examples section below, murine norovirus (MNV) infection causes γδ T cells in the gut to be replaced by TNFα-secreting T cells in a preclinical animal model of Crohn's disease. To investigate this process further, an ex vivo platform was utilized in which enteroids are cultured together with immune cells. Remarkably, co-culturing anti-inflammatory T cells with murine Atg16L1^{-/-} enteroids blocks necroptosis and restores Paneth cells. Mass spectrometry of the culture supernatant identified API5, a protein previously not known to be secreted by lymphocytes or have a role in the intestinal barrier. Depleting API5 with an antibody inhibited protection mediated by γδ T cells, and an addition of recombinant human API5 (rhAPI5) to the media was sufficient to protect enteroids from TNFα-induced death (FIG. 6).

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art.

The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

The following terms, unless otherwise indicated, shall be understood to have the following meanings:

The terms “polypeptide” and “protein” used interchangeably herein encompass native or artificial proteins, protein fragments and polypeptide analogs of a protein sequence. A polypeptide or protein may be monomeric or polymeric.

The term “isolated protein” or “isolated polypeptide” is a protein or polypeptide that by virtue of its origin or source of derivation has one to four of the following: (1) is not associated with naturally associated components that accompany it in its native state, (2) is free of other proteins from the same species, (3) is expressed by a cell from a different species, or (4) does not occur in nature. Thus, a polypeptide or protein that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art.

The term “fragment” in regard to polypeptides refers to a polypeptide that has an amino-terminal and/or carboxy-terminal deletion, but where the remaining amino acid sequence is identical to the corresponding positions in the full-length naturally-occurring sequence. Also, fragments according to the invention may be made by truncation, e.g., by removal of one or more amino acids from the N and/or C-terminal ends of a polypeptide. Up to 10, up to 20, up to 30, up to 40 or more amino acids may be removed from the N and/or C terminal in this way. Fragments may also be generated by one or more internal deletions. In some embodiments, fragments are at least 5, 6, 8 or 10 amino acids long. In other embodiments, the fragments are at least 14, at least 20, at least 50, or at least 70, 80, 90, 100, 150, 200, or 400 amino acids long.

In certain embodiments, amino acid substitutions of a protein or portion thereof are those which: (1) reduce susceptibility to proteolysis, (2) reduce susceptibility to oxidation, (3) alter binding affinity for forming protein complexes, or (4) confer or modify other physicochemical or functional properties. For example, single or multiple amino acid substitutions (preferably conservative amino acid substitutions) may be made in the normally-occurring sequence.

A conservative amino acid substitution should not substantially change the structural characteristics of the parent sequence. Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al., Nature 354:105 (1991), which are each incorporated herein by reference.

As used herein, the twenty naturally occurring amino acids and their abbreviations follow conventional usage. See Immunology—A Synthesis (2^{nd} Edition, E. S. Golub and D. R. Gren, Eds., Sinauer Associates, Sunderland, Mass. (1991)), which is incorporated herein by reference.

The term “polynucleotide” as referred to herein means a polymeric form of nucleotides of at least 10 bases in length, either ribonucleotides or deoxyribonucleotides or a modified form of either type of nucleotide. The term includes single and double stranded forms.

The term “isolated polynucleotide” as used herein means a polynucleotide of genomic, cDNA, or synthetic origin or some combination thereof, which by virtue of its origin or source of derivation, the “isolated polynucleotide” has one to three of the following: (1) is not associated with all or a portion of a polynucleotides with which the “isolated polynucleotide” is found in nature, (2) is operably linked to a polynucleotide to which it is not linked in nature, or (3) does not occur in nature as part of a larger sequence.

The term “oligonucleotide” as used herein includes naturally occurring, and modified nucleotides linked together by naturally occurring and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g. for primers and probes; although oligonucleotides may be double stranded, e.g. for use in the construction of a gene mutant. Oligonucleotides of the invention can be either sense or antisense oligonucleotides.

The term “naturally occurring nucleotides” as used herein includes deoxyribonucleotides and ribonucleotides. The term “modified nucleotides” as used herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al., Nucl. Acids Res. 14:9081 (1986); Stec et al., J. Am. Chem. Soc. 106:6077 (1984); Stein et al., Nucl. Acids Res. 16:3209 (1988); Zon et al., Anti-Cancer Drug Design 6:539 (1991); Zon et al., Oligonucleotides and Analogues: A Practical Approach, pp. 87-108 (F. Eckstein, Ed., Oxford University Press, Oxford England (1991)); U.S. Pat. No. 5,151,510; Uhlmann and Peyman, Chemical Reviews 90:543 (1990), the disclosures of which are hereby incorporated by reference. An oligonucleotide can include a label for detection, if desired.

“Operably linked” sequences include both expression control sequences that are contiguous with the gene of interest and expression control sequences that act in trans or at a distance to control the gene of interest. The term “expression control sequence” as used herein means polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “vector”, as used herein, means a nucleic acid molecule capable of transporting another nucleic acid to which it has been linked. In some embodiments, the vector is a plasmid, i.e., a circular double stranded DNA loop into which additional DNA segments may be ligated. In some embodiments, the vector is a viral vector, wherein additional DNA segments may be ligated into the viral genome. In some embodiments, the vectors are capable of autonomous replication in a host cell into which they are introduced (e.g., bacterial vectors having a bacterial origin of replication and episomal mammalian vectors). In other embodiments, the vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”).

The term “promoter” as used herein is defined as a DNA sequence recognized by the synthetic machinery of the cell, or introduced synthetic machinery, required to initiate the specific transcription of a polynucleotide sequence. As used herein, the term “regulatory sequence” means a nucleic acid sequence which can regulate expression of a gene product operably linked to the regulatory sequence. In some instances, this sequence may be the core promoter sequence and in other instances, this sequence may also include an enhancer sequence and other regulatory elements which are required for expression of the gene product. The promoter or regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

The term “recombinant host cell” (or simply “host cell”), as used herein, means a cell into which an exogenous nucleic acid and/or recombinant vector has been introduced. It should be understood that “recombinant host cell” and “host cell” mean not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein.

The term “percent sequence identity” means a ratio, expressed as a percent of the number of identical residues over the number of residues compared.

Sequence identity for nucleic acid sequences may be analyzed over a stretch of at least about nine nucleotides, usually at least about 18 nucleotides, more usually at least about 24 nucleotides, typically at least about 28 nucleotides, more typically at least about 32 nucleotides, and preferably at least about 36, 48 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA, which includes, e.g., the programs FASTA2 and FASTA3, provides alignments and percent sequence identity of the regions of the best overlap between the query and search sequences (Pearson, Methods Enzymol. 183:63-98 (1990); Pearson, Methods Mol. Biol. 132:185-219 (2000); Pearson, Methods Enzymol. 266:227-258 (1996); Pearson, J. Mol. Biol. 276:71-84 (1998); herein incorporated by reference). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference.

A reference to a nucleotide sequence encompasses its complement unless otherwise specified. Thus, a reference to a nucleic acid having a particular sequence should be understood to encompass its complementary strand, with its complementary sequence.

Sequence identity for polypeptides, is typically measured using sequence analysis software. Protein analysis software matches sequences using measures of similarity assigned to various substitutions, deletions and other modifications, including conservative amino acid substitutions. For instance, GCG contains programs such as “Gap” and “Bestfit” which can be used with default parameters, as specified with the programs, to determine sequence homology or sequence identity between closely related polypeptides, such as homologous polypeptides from different species of organisms or between a wild-type protein and a mutein thereof. See, e.g., GCG Version 6.1. Polypeptide sequences also can be compared using FASTA using default or recommended parameters, see GCG Version 6.1. (University of Wisconsin Wis.) FASTA (e.g., FASTA2 and FASTA3) provides alignments and percent sequence identity of the regions of the best overlap between the query and search sequences (Pearson, Methods Enzymol. 183:63-98 (1990); Pearson, Methods Mol. Biol. 132:185-219 (2000)). Another preferred algorithm when comparing a sequence of the invention to a database containing a large number of sequences from different organisms is the computer program BLAST, especially blastp or tblastn, using default parameters, as supplied with the programs. See, e.g., Altschul et al., J Mol. Biol. 215:403-410 (1990); Altschul et al., Nucleic Acids Res. 25:3389-402 (1997).

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences.

The term “substantial similarity” or “substantial sequence similarity,” when referring to a nucleic acid or fragment thereof, means that when optimally aligned with appropriate nucleotide insertions or deletions with another nucleic acid (or its complementary strand), there is nucleotide sequence identity in at least about 85%, preferably at least about 90%, 91%, 92%, 93%, 94%, 95%, 96%, 97%, 98% or 99% of the nucleotide bases, as measured by any well-known algorithm of sequence identity, such as FASTA, BLAST or Gap, as discussed above.

As applied to polypeptides, the term “substantial identity” means that two peptide sequences, when optimally aligned, such as by the programs GAP or BESTFIT using default gap weights, as supplied with the programs, share at least 70%, 75%, 80% or 85% sequence identity, preferably at least 90%, 91%, 92%, 93%, 94% 95%, 96%, 97%, 98% or 99% sequence identity. In certain embodiments, residue positions that are not identical differ by conservative amino acid substitutions.

A “conservative amino acid substitution” is one in which an amino acid residue is substituted by another amino acid residue having a side chain R group with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity may be adjusted upwards to correct for the conservative nature of the substitution. Means for making this adjustment are well-known to those of skill in the art. See, e.g., Pearson, Methods Mol. Biol. 243:307-31 (1994). Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups are: valine-leucine-isoleucine, phenylalanine-tyrosine, lysine-arginine, alanine-valine, glutamate-aspartate, and asparagine-glutamine.

Alternatively, a conservative substitution or replacement, as the terms are used interchangeably herein, is any change having a positive value in the PAM250 log-likelihood matrix disclosed in Gonnet et al., Science 256:1443-45 (1992), herein incorporated by reference. A “moderately conservative” replacement is any change having a nonnegative value in the PAM250 log-likelihood matrix.

The term “potency” is a measurement of biological activity and may be designated as IC₅₀, or effective concentration of a protein needed to inhibit 50% of a biological activity in a cell which activity is mediated by the protein.

The phrase “effective amount” or “therapeutically effective amount” as used herein refers to an amount necessary (at dosages and for periods of time and for the means of administration) to achieve the desired therapeutic result. An effective amount is at least the minimal amount, but less than a toxic amount, of an active agent which is necessary to impart therapeutic benefit to a subject.

By “IgG” as used herein is meant a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3. By “immunoglobulin (Ig)” herein is meant a protein consisting of one or more polypeptides substantially encoded by immunoglobulin genes. Immunoglobulins include but are not limited to antibodies. Immunoglobulins may have a number of structural forms, including but not limited to full length antibodies, antibody fragments, and individual immunoglobulin domains. By “immunoglobulin (Ig) domain” herein is meant a region of an immunoglobulin that exists as a distinct structural entity as ascertained by one skilled in the art of protein structure. Ig domains typically have a characteristic folding topology. The known Ig domains in the IgG class of antibodies are the variable heavy chain domain (VH), the heavy chain constant domains-Cγ1, Cy2, Cy3-together comprising the Cγ domain which includes the hinge region between Cγ1 and Cy2, the variable domain of the light chain (VL), and the constant domain of the light chain (CL), which in humans comprises either the kappa (CO or lambda (CA) light chain constant domain.

As known in the art, the term “Fc region” is used to define a C-terminal region of an immunoglobulin heavy chain. The “Fc region” (also known as the “fragment crystallizable” or “tail” region) may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. For all heavy chain constant region amino acid positions discussed in the present invention, numbering is according to the EU index first described in Edelman et al., 1969, Proc. Nat. Acad. Sci. USA 63(1):78-85, describing the amino acid sequence of myeloma protein EU, which is the first human IgG1 sequenced. The EU index of Edelman et al. is also set forth in Kabat et al., Sequences of Proteins of Immunological Interest, 5th Ed. Public Health Service, National Institutes of Health, Bethesda, Md., 1991. Thus, the “EU index as set forth in Kabat” or “EU index of Kabat” refers to the amino acid residue numbering system based on the human IgG1 EU antibody of Edelman et al. as set forth in Kabat 1991.

The Fc region of an immunoglobulin generally comprises two constant domains, CH2 and CH3. Typically, an “Fc polypeptide,” as the term is used herein, comprises a CH2 and a CH3 domain and can include at least a portion of the hinge domain, but does not usually include the entire CH1 domain. As is known in the art, an Fc region can be present in dimeric or monomeric form.

As used in the art, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, 1991, Ann. Rev. Immunol., 9:457-92; Capel et al., 1994, Immunomethods, 4:25-34; and de Haas et al., 1995, J Lab. Clin. Med., 126:330-41. “FcR” also includes the neonatal receptor, FcRn, which is responsible for the transfer of maternal IgGs to the fetus (Guyer et al., 1976, J Immunol., 117:587; and Kim et al., 1994, J Immunol., 24:249).

As used herein, “pharmaceutically acceptable carrier” or “pharmaceutical acceptable excipient” includes any material which, when combined with an active ingredient, allows the ingredient to retain biological activity and is non-reactive with the subject's immune system. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

The term “treating”, as used herein, unless otherwise indicated, means reversing, alleviating, inhibiting the progress of, delaying the progression of, delaying the onset of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject. For the avoidance of doubt, reference herein to “treatment” includes reference to curative, palliative and prophylactic treatment.

### Polypeptides, Polynucleotides and Vectors

In one aspect, the present disclosure provides a recombinant protein comprising an apoptosis inhibitor 5 (API5) protein, or a fragment or a variant thereof.

In some embodiments, the API5 protein comprises the amino acid sequence of SEQ ID NO: 1, or a sequence having at least 70%, 80%, 85%, 90%, 91%, 92%, 93%, 94%, 94%, 95%, 96%, 97%, 98%, or 99% identity thereto.

In some embodiments, the API5 protein comprises the amino acid sequence of SEQ ID NO: 2, or a sequence having at least 70%, 80%, 85%, 90%, 91%, 92%, 93%, 94%, 94%, 95%, 96%, 97%, 98%, or 99% identity thereto.

Human API5
>sp|Q9BZZ5|API5_HUMAN
(SEQ ID NO: 1)
MPTVEELYRNYGILADATEQVGQHKDAYQVILDGVKGGTKEKRLAAQFIPKFFKHFPELA
DSAINAQLDLCEDEDVSIRRQAIKELPQFATGENLPRVADILTQLLQTDDSAEENLVNNA
LLSIFKMDAKGTLGGLESQILQGEDIVRERAIKELSTKLKTLPDEVLTKEVEELILTESK
KVLEDVTGEEFVLEMKILSGLKSLQTVSGRQQLVELVAEQADLEQTFNPSDPDCVDRLLQ
CTRQAVPLESKNVHSTRFVTYFCEQVLPNLGTLTTPVEGLDIQLEVLKLLAEMSSFCGDM
EKLETNLRKLFDKLLEYMPLPPEEAENGENAGNEEPKLQFSYVECLLYSFHQLGRKLPDF
LTAKLNAEKLKDFKIRLQYFARGLQVYIRQLRLALQGKTGEALKTEENKIKVVALKITNN
INVLIKDLFHIPPSYKSTVTLSWKPVQKVEIGQKRASEDTTSGSPPKKSSAGPKRDARQI
YNPPSGKYSSNLGNFNYEQRGAFRGSRGGRGWGTRGNRSRGRLY
Mouse API5
>sp|O35841|API5_MOUSE
(SEQ ID NO: 2)
MPTVEELYRNYGILADATEQVGQHKDAYQVILDGVKGGTKEKRLAAQFIPKFFKHFPELA
DSAINAQLDLCEDEDVSIRRQAIKELPQFATGENLPRVADILTQLLQTDDSAEENLVNNA
LLSIFKMDAKGTLGGLESQILQGEDIVRERAIKFLSTKLKTLPDEVLTKEVEELILTESK
KVLEDVTGEEFVLFMKILSGLKSLQTVSGRQQLVELVAEQADLEQAFSPSDPDCVDRLLQ
CTRQAVPLESKNVHSTRFVTYFCEQVLPNLSTLTTPVEGLDIQLEVLKLLAEMSSFCGDM
EKLETNLRKLFDKLLEYMPLPPEEAENGENAGNEEPKLQFSYVECLLYSFHQLGRKLPDF
LTAKLNAEKLKDFKIRLQYFARGLQVYIRQLRLALQGKTGEALKTEENKIKVVALKITNN
INVLIKDLFHIPPSYKSTVTLSWKPVQKVEIGQKRTSEDTSSGSPPKKSPGGPKRDARQI
YNPPSGKYSSNLSNFNYERSLQGK

In some embodiments, the recombinant protein comprises a fragment of the API5 protein.

In some embodiments, the API5 protein fragment comprises the N-terminal HEAT repeat region of API5. This fragment corresponds to the N-terminal HEAT repeat region of API5 (Han et al. J Biol Chem, 287:10727 (2012), which is incorporated herein by reference in its entirety for all purposes). For example, the fragment of API5 comprises residues 1-206 of SEQ ID NO: 1. In one embodiment, the API5 protein fragment comprises the amino acid sequence of SEQ ID NO: 8, or a sequence having at least 70%, 80%, 85%, 90%, 91%, 92%, 93%, 94%, 94%, 95%, 96%, 97%, 98%, or 99% identity thereto.

residues 1-206 of human API5
(SEQ ID NO: 8)
MPTVEELYRNYGILADATEQVGQHKDAYQVILDGVKGGTKEKRLAAQFIPKFFKHFPELA
DSAINAQLDLCEDEDVSIRRQAIKELPQFATGENLPRVADILTQLLQTDDSAEFNLVNNA
LLSIFKMDAKGTLGGLESQILQGEDIVRERAIKFLSTKLKTLPDEVLTKEVEELILTESK
KVLEDVTGEEFVLEMKILSGLKSLQT

In some embodiments, the API5 protein fragment comprises residues 1-448 of SEQ ID NO: 1. In one embodiment, the API5 protein fragment comprises the amino acid sequence of SEQ ID NO: 7, or a sequence having at least 70%, 80%, 85%, 90%, 91%, 92%, 93%, 94%, 94%, 95%, 96%, 97%, 98%, or 99% identity thereto.

(SEQ ID NO: 7)
residues 1-448 of human API5
MPTVEELYRNYGILADATEQVGQHKDAYQVILDGVKGGTKEKRLAAQFIPKFFKHFPELA
DSAINAQLDLCEDEDVSIRRQAIKELPQFATGENLPRVADILTQLLQTDDSAEFNLVNNA
LLSIFKMDAKGTLGGLESQILQGEDIVRERAIKELSTKLKTLPDEVLTKEVEELILTESK
KVLEDVTGEEFVLEMKILSGLKSLQTVSGRQQLVELVAEQADLEQTFNPSDPDCVDRLLQ
CTRQAVPLESKNVHSTRFVTYFCEQVLPNLGTLTTPVEGLDIQLEVLKLLAEMSSFCGDM
EKLETNLRKLFDKLLEYMPLPPEEAENGENAGNEEPKLQFSYVECLLYSFHQLGRKLPDF
LTAKLNAEKLKDFKIRLQYFARGLQVYIRQLRLALQGKTGEALKTEENKIKVVALKITNN
INVLIKDLFHIPPSYKSTVTLSWKPVQK

In some embodiments, the API5 protein, or a fragment or a variant thereof, is genetically fused to and/or chemically conjugated to one or more heterologous moieties.

Heterologous moieties suitable for genetical fusion and/or chemical conjugation with the API5 protein, or a fragment or a variant thereof, include, but are not limited to, peptides, polypeptides, small molecules, polymers, nucleic acids, lipids, sugars, etc.

Heterologous peptides and polypeptides include, but are not limited to, an epitope (e.g., FLAG) or a tag sequence (e.g., His₆ (SEQ ID NO: 78), and the like) to allow for the detection and/or isolation of a recombinant API5 protein; a transmembrane receptor protein or a portion thereof, such as an extracellular domain or a transmembrane and intracellular domain; a ligand or a portion thereof which binds to a transmembrane receptor protein; an enzyme or portion thereof which is catalytically active; a polypeptide or peptide which promotes oligomerization, such as a leucine zipper domain; a polypeptide or peptide which increases stability, such as an immunoglobulin constant region (e.g., an Fc domain); a half life-extending sequence comprising a combination of two or more (e.g., 2, 5, 10, 15, 20, 25, etc) naturally occurring or non-naturally occurring charged and/or uncharged amino acids (e.g., Serine, Glycine, Glutamic or Aspartic Acid) designed to form a predominantly hydrophilic or predominantly hydrophobic fusion partner for a recombinant API5 protein; a functional or non-functional antibody, or a heavy or light chain thereof, and a polypeptide which has an activity, such as a therapeutic activity, different from recombinant API5 proteins of the present invention.

In some embodiments, the one or more heterologous moieties comprise one or more affinity tags. Non-limiting examples of affinity tag suitable to be used in the present disclosure include a His tag, an Avi-tag, a hemagglutinin (HA) tag, a FLAG tag, a Myc tag, a GST tag, a MBP tag, a chitin binding protein tag, a calmodulin tag, a V5 tag, a streptavidin binding tag, a green fluorescent protein (GFP), YFP, RFP, CFP, mCherry, tdTomato, SUMO tag, Ubiquitin tag, and a combination thereof. In one embodiment, the one or more heterologous moieties comprise a His₆ tag (SEQ ID NO: 78) and an Avi-tag and optionally comprise the amino acid sequence of MKHHHHHHSSGLNDIFEAQKIEWHE (SEQ ID NO: 9).

In some embodiments, the recombinant API5 proteins of the present disclosure further comprise a protease cleavage site between the API5 protein, or a fragment or a variant thereof, and the one or more affinity tags. In one embodiment, the protease cleavage site is a cleavage site for the TEV protease and optionally comprises the amino acid sequence of ENLYFQGS (SEQ ID NO: 10).

In one embodiment, the recombinant API5 protein comprises the amino acid sequence of SEQ ID NO: 3, or a sequence having at least 70%, 80%, 85%, 90%, 91%, 92%, 93%, 94%, 94%, 95%, 96%, 97%, 98%, or 99% identity thereto.

In one embodiment, the recombinant API5 protein comprises the amino acid sequence of SEQ ID NO: 6, or a sequence having at least 70%, 80%, 85%, 90%, 91%, 92%, 93%, 94%, 94%, 95%, 96%, 97%, 98%, or 99% identity thereto.

In some embodiments, the one or more heterologous moieties comprise a moiety that specifically binds albumin. Linkage to moieties that bind albumin has been shown to extend the half-life of short lived proteins. The moiety that specifically binds albumin may be a small molecule, a peptide, a polypeptide, a lipid, etc. The albumin may be rat albumin, rabbit albumin, or human albumin. In some embodiments, the albumin is human serum albumin.

In some embodiments, the moiety that specifically binds albumin comprises a albumin-binding peptide described in the art, for example, in Dennis et al., J Biol Chem. 2002 Sep. 20; 277(38):35035-43 and U.S. Pat. No. 10,442,851, the content of both of which is incorporated herein by reference in its entirety for all purposes. In some embodiments, the moiety that specifically binds albumin comprises the amino acid sequence of any one of SEQ ID NOs: 12-66, 76 and 77.

Additional albumin-binding moieties that are suitable for use in the present disclosure include those described in Zorzi et al., Medchemcomm. 2019 Jun. 6; 10(7):1068-1081, which is incorporated herein by reference in its entirety for all purposes. In some embodiments, the moiety that specifically binds albumin is Naphthalene acylsulfonamide, Diphenylcyclohexanol phosphate ester, 9-fluorenylmethoxycarbonyl (Fmoc), Fmoc derivative linked to a 16-sulfanylhexadecanoic acid through a maleimide group, Dicoumarol derivative with maleimide, Evans blue derivative with maleimide, Diflunisal-γGlu-LysIJ±O2O c)-indomethacin, lithocholic acid coupled to a γGlu linker, 6-(4-(p-Iodophenyl) butanamido) hexanoate, A083/B134, A099/B344, 89D03 (Ac-WWEQDRDWDFDVFGGGTP-NH₂, SEQ ID NO: 67), acylated heptapeptide F-tag (fluorescein-EYEK(palmitate)EYE-NH₂, SEQ ID NO: 68); disulfide cyclized peptide SA21 (Ac-RLIEDICLPRWGCLWEDD-NH₂, SEQ ID NO: 69), head-to-tail cyclized peptide HSA-1 (AK*K*PGK*AK*PG with variable lysine (K*), SEQ ID NO: 70), ABD035, ABDCon, DARPins, AlbudAbs, dsFv CA645, Nanobody Nb.b201, or VNAR E06.

In some embodiments, the API5 proteins, or fragments or variants thereof, are fused to an Fc domain, e.g., one or more domains of an Fc region of a human IgG. Antibodies comprise two functionally independent parts, a variable domain known as “Fab,” that binds an antigen, and a constant domain known as “Fc,” that is involved in, among other things, effector functions such as complement activation and attack by phagocytic cells. An Fc has a long serum half-life (Capon et al., 1989, Nature 337: 525-31) such that when joined together with a therapeutic protein, an Fc domain can provide longer half-life or incorporate such effector functions as Fc receptor binding, protein A binding, complement fixation, and other characteristics that are desirable in a therapeutic protein.

Fc sequences may be fused to the API5 proteins disclosed herein, or fragments or variants thereof, to extend the half-life of the API5 proteins. In some embodiments, the Fc domain may be modified to alter effector function of the domain. In some embodiments, the Fc domain is modified to enhance the half-life of the recombinant protein. Modification of IgG1 Fc may be performed as described in the art, for example in U.S. Pat. No. 10,464,979, which is incorporated herein by reference in its entirety for all purposes.

Table 1 below illustrates some of the Fc modifications exemplified in this application. In some embodiments, an Fc domain used for fusion with the API5 proteins disclosed herein does not include the C-terminal Lys residue.

TABLE 1
Human IgG1 Fc Sequences

| | | SEQ ID |
|---|---|---|
| Description | Amino Acid Sequence | NO: |
| Human Wild- | EPKSCDKTHTCPPCPAPELLGGPSVFLEPPKPKDTLMISRT | 71 |
| Type IgG1 Fc | PEVTCVVVDVSHEDPEVKFNWYVDGVEVHNAKTKPREE | |
| (Bold and | QYNSTYRVVSVLTVLHQDWLNGKEYKCKVSNKALPAPIE | |
| underlined | KTISKAKGQPREPQVYTLPPSREEMTKNQVSLTCLVKGFY | |
| residues are | PSDIAVEWESNGQPENNYKTTPPVLDSDGSFFLYSKLTVD | |
| modified in | KSRWQQGNVFSCSVMHEALHNHYTQKSLSLSPGK | |
| construct below) | | |
| Human IgG1 | EPKSCDKTHTCPPCPAPEAAGAPSVFLFPPKPKDTLMISRT | 72 |
| FC1 | PEVTCVVVDVSHEDPEVKFNWYVDGVEVHNAKTKPREE | |
| | QYNSTYRVVSVLTVLHQDWLNGKEYKCKVSNKALPAPIE | |
| | KTISKAKGQPREPQVYTLPPSREEMTKNQVSLTCLVKGFY | |
| | PSDIAVEWESNGQPENNYKTTPPVLDSDGSFFLYSKLTVD | |
| | KSRWQQGNVFSCSVLHEALHSHYTQKSLSLSPGK | |

In some embodiments, the API5 protein, or a fragment or a variant thereof, may be fused to other large long-lived proteins such as albumin (Syed et al., Blood (1997) 89, 3243-3252; Yeh et al., Proc. Natl. Acad. Sci. U.S.A (1992) 89, 1904-1908; the content of each of which is incorporated herein by reference in its entirety).

Other suitable modifications to the API5 recombinant protein include introduction of glycosylation sites (Keyt et al., Proc. Natl. Acad. Sci. U.S.A (1994) 91, 3670-3674, the content of each is incorporated herein by reference in its entirety), and conjugation with polyethylene glycol polymers (i.e. PEG) (Clark et al., J. Biol. Chem. (1996) 271, 21969-21977; Lee et al., Bioconjugate Chem. (1999) 10, 973-981; Tanaka et al., Cancer Res. (1991) 51, 3710-3714; the content of each of which is incorporated herein by reference in its entirety).

In some embodiments, recombinant API5 proteins can be made by fusing heterologous sequences at either the N-terminus or at the C-terminus of the API5 protein, or a fragment or a variant thereof. As described herein, a heterologous sequence can be an amino acid sequence (e.g., albumin-binding peptides/proteins, Fc domains) or a non-amino acid-containing polymer (e.g., PEG). Heterologous sequences can be fused either directly to the API5 protein, or a fragment or a variant thereof, either chemically or by recombinant expression from a single polynucleotide or they may be joined via a linker or adapter molecule. A peptidyl linker or adapter molecule can be one or more amino acid residues (or -mers), e.g., 1, 2, 3, 4, 5, 6, 7, 8, or 9 residues (or -mers), preferably from 10 to 50 amino acid residues (or -mers), e.g., 10, 11, 12, 13, 14, 15, 16, 17, 18, 19, 20, 25, 30, 35, 40, 45, or 50 residues (or -mers), and more preferably from 15 to 35 amino acid residues (or -mers). A linker or adapter molecule can also be designed with a cleavage site for a protease to allow for the separation of the fused moieties. Non-limiting examples of non-amino acid-containing polymers include poly (ethylene glycol) (PEG), poly (propylene glycol), copolymers of ethylene glycol and propylene glycol, polyoxy ethylated polyols, polyvinyl alcohols, polysaccharides, dextran, polyvinyl ethers, biodegradable polymers such as PLA (poly (lactic acid)) and PLGA (poly (lactic-glycolic acid)), lipid polymers, chitin, hyaluronic acid, and the like.

When forming the recombinant proteins of the present invention, a linker can, but need not, be employed. The linker can be made up of amino acids linked together by peptide bonds, i.e., a peptidyl linker. In some embodiments of the present invention, the linker is made up of from 1 to 20 or more amino acids linked by peptide bonds, wherein the amino acids are selected from the 20 naturally occurring amino acids. In some embodiments, the amino acids are selected from the amino acids glycine, serine, and glutamate. In some embodiments, suitable linkers include, for example, GSGEGEGSEGSG (SEQ ID NO: 73); GGSEGEGSEGGS (SEQ ID NO: 74); and GGGS (SEQ ID NO: 79). The present invention contemplates linkers of any length or composition. Exemplary linkers are shown in Table 2.

TABLE 2
Linker Sequences

| Description | Sequence | SEQ ID NO: |
|---|---|---|
| Linker L1 | GSGEGEGSEGSG | 73 |
| Linker L2 | GGSEGEGSEGGS | 74 |
| Linker L3 | GGGGS | 75 |
| Linker L4 | GGGS | 79 |

The linkers described herein are exemplary, and linkers that are much longer and which include other residues are also contemplated by the present invention.

In one aspect, the present disclosure provides an isolated polynucleotide encoding the recombinant API5 protein described herein. For expression of a polynucleotide described herein, a promoter sequence may be included to position the start site for RNA synthesis. The promoter may be a constitutive promoter or inducible promoter. The polynucleotide may also be operably linked to one or more additional regulatory sequences, such as terminators or enhancers. In one embodiment, the isolated polynucleotide is an mRNA.

In one aspect, the present disclosure provides a vector comprising the isolated polynucleotide encoding the recombinant protein described herein.

In order to assess the expression of a recombinant API5 protein described herein, a polynucleotide or vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

In one aspect, the present disclosure provides a host cell comprising the isolated polynucleotide or the vectors described herein.

### Pharmaceutical Compositions

Pharmaceutical compositions comprising the recombinant API5 proteins, polynucleotides, or vectors described herein are within the scope of the present invention. Such pharmaceutical compositions can comprise a therapeutically effective amount of a recombinant API5 protein, polynucleotide, or vector, in admixture with a pharmaceutically or physiologically acceptable formulation agent selected for suitability with the mode of administration. Acceptable formulation agents preferably are nontoxic to recipients at the dosages and concentrations employed.

The pharmaceutical composition can contain formulation agent(s) for modifying, maintaining, or preserving, for example, the pH, osmolarity, viscosity, clarity, color, isotonicity, odor, sterility, stability, rate of dissolution or release, adsorption, or penetration of the composition. Suitable formulation agents include, but are not limited to, amino acids (such as glycine, glutamine, asparagine, arginine, or lysine), antimicrobials, antioxidants (such as ascorbic acid, sodium sulfite, methionine or sodium hydrogen-sulfite), buffers (such as borate, bicarbonate, Tris-HCl, histidine, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants (see, e.g., Remington's Pharmaceutical Sciences (18th Ed., A. R. Gennaro, ed., Mack Publishing Company 1990), and subsequent editions of the same, incorporated herein by reference for any purpose).

The optimal pharmaceutical composition will be determined by a skilled artisan depending upon, for example, the intended route of administration, delivery format, and desired dosage (see, e.g., Remington's Pharmaceutical Sciences, supra). Such compositions can influence the physical state, stability, rate of in vivo release, and rate of in vivo clearance of the recombinant API5 protein, polynucleotide, or vector.

The primary vehicle or carrier in a pharmaceutical composition can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Histidine or Tris buffer of about pH 6.0-8.5, which can further include sorbitol or a suitable substitute. In one embodiment of the present invention, recombinant API5 protein compositions can be prepared for storage by mixing the selected composition having the desired degree of purity with optional formulation agents (Remington's Pharmaceutical Sciences, supra) in the form of an aqueous solution.

The pharmaceutical compositions can be selected for parenteral delivery. Alternatively, the compositions can be selected for inhalation or for delivery through the digestive tract, such as orally. The preparation of such pharmaceutically acceptable compositions is within the skill of the art. The formulation components are present in concentrations that are acceptable to the site of administration. For example, buffers are used to maintain the composition at physiological pH or at a slightly lower pH, typically within a pH range of from about 6 to about 8.

When parenteral administration is contemplated, the therapeutic compositions for use in this invention can be in the form of a pyrogen-free, parenterally acceptable, aqueous solution comprising the desired recombinant API5 protein, polynucleotide, or vector, in a pharmaceutically acceptable vehicle. A particularly suitable vehicle for parenteral injection is sterile distilled water in which a recombinant API5 protein, polynucleotide, or vector, is formulated as a sterile, isotonic solution, properly preserved. Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which can then be delivered via a depot injection. Hyaluronic acid can also be used, and this can have the effect of promoting sustained duration in the circulation. Other suitable means for the introduction of the desired molecule include implantable drug delivery devices.

In one embodiment, a pharmaceutical composition can be formulated for inhalation. For example, the pharmaceutical composition can be formulated as a dry powder for inhalation. Inhalation solutions can also be formulated with a propellant for aerosol delivery. In yet another embodiment, solutions can be nebulized. Pulmonary administration is further described in International Publication No. WO1994020069, which describes the pulmonary delivery of chemically modified proteins.

It is also contemplated that certain formulations can be administered orally. In one embodiment of the present invention, formulations that are administered in this fashion can be formulated with or without those carriers customarily used in the compounding of solid dosage forms such as tablets and capsules. For example, a capsule can be designed to release the active portion of the formulation at the point in the gastrointestinal tract when bioavailability is maximized and pre-systemic degradation is minimized. Additional agents can be included to facilitate absorption. Diluents, flavorings, low melting point waxes, vegetable oils, lubricants, suspending agents, tablet disintegrating agents, and binders can also be employed.

Another pharmaceutical composition can involve an effective quantity of recombinant API5 protein in a mixture with non-toxic excipients that are suitable for the manufacture of tablets. By dissolving the tablets in sterile water, or another appropriate vehicle, solutions can be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving recombinant API5 proteins, polynucleotides, or vectors, in sustained- or controlled-delivery formulations. Techniques for formulating a variety of other sustained- or controlled-delivery means, such as liposome carriers, bio-erodible microparticles or porous beads and depot injections, are also known to those skilled in the art (see, e.g., International Publication No. WO1993015722, which describes the controlled release of porous polymeric microparticles for the delivery of pharmaceutical compositions, and Wischke & Schwendeman, 2008, Int. J. Pharm. 364: 298-327, and Freiberg & Zhu, 2004, Int. J. Pharm. 282: 1-18, which discuss microsphere/microparticle preparation and use). As described herein, a hydrogel is an example of a sustained- or controlled-delivery formulation.

Additional examples of sustained-release preparations include semipermeable polymer matrices in the form of shaped articles, e.g. films, or microcapsules. Sustained release matrices can include polyesters, hydrogels, polylactides (U.S. Pat. No. 3,773,919 and European Patent No. 0 058 481), copolymers of L-glutamic acid and gamma ethyl-L-glutamate (Sidman et ah, 1983, Biopolymers 22: 547-56), poly(2-hydroxyethyl-methacrylate) (Langer et ah, 1981, J Biomed. Mater. Res. 15: 167-277 and Langer, 1982, Chem. Tech. 12: 98-105), ethylene vinyl acetate (Langer et al, supra) or poly-D(−)-3-hydroxybutyric acid (European Patent No. 0133988). Sustained-release compositions can also include liposomes, which can be prepared by any of several methods known in the art. See, e.g., Epstein et ah, 1985, Proc. Natl. Acad. Sci. U.S.A. 82: 3688-92; and European Patent Nos. 0036676, 0088046, and 0143949.

The pharmaceutical composition to be used for in vivo administration typically should be sterile. This can be accomplished by filtration through sterile filtration membranes. Where the composition is lyophilized, sterilization using this method can be conducted either prior to, or following, lyophilization and reconstitution. The composition for parenteral administration can be stored in lyophilized form or in a solution. In addition, parenteral compositions generally are placed into a container having a sterile access port, for example, an intravenous solution bag or vial having a stopper pierceable by a hypodermic injection needle. The parenteral composition can be diluted into parenteral acceptable diluents (e.g., saline and 5% Dextrose).

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration.

In one embodiment, the present invention is directed to kits for producing a single-dose administration unit. The kits can each contain both a first container having a dried protein and a second container having an aqueous formulation. Also included within the scope of this invention are kits containing single and multi-chambered pre-filled syringes (e.g., liquid syringes and dual chamber syringes).

In one embodiment, the present invention is directed to a pharmaceutical composition comprising a recombinant API5 protein formulated as a powder for injection after reconstitution to a solution for injection.

Selecting an administration regimen for a therapeutic depends on several factors, including the serum or tissue turnover rate of the entity, the level of symptoms, the immunogenicity of the entity, and the accessibility of the target cells in the biological matrix. In certain embodiments, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, Fc fusion therapeutic proteins, cytokines, and small molecules are available (see, e.g., Wawrzynczak, 1996, Antibody Therapy, Bios Scientific Pub. Ltd, Oxfordshire, UK; Kresina (ed.), 1991, Monoclonal Antibodies, Cytokines and Arthritis, Marcel Dekker, New York, N.Y.; Bach (ed.), 1993, Monoclonal Antibodies and Peptide Therapy in Autoimmune Diseases, Marcel Dekker, New York, N.Y.; Baert, et al., 2003, New Engl. J. Med. 348:601-608; Milgrom, et al., 1999, New Engl. J. Med. 341:1966-1973; Slamon, et al., 2001, New Engl. J. Med. 344:783-792; Beniaminovitz, et al., 2000, New Engl. J Med. 342:613-619; Ghosh, et al., 2003, New Engl. J Med. 348:24-32; Lipsky, et al., 2000, New Engl. J Med. 343:1594-1602).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., increased serum phosphate or decreased phosphate excretion.

Actual dosage levels of the active ingredients in the pharmaceutical compositions of the present disclosure may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

Compositions comprising the recombinant API5 proteins of the disclosure can be provided by continuous infusion, or by doses at intervals of, e.g., one day, one week, 1-7 times per week, or one month. Doses may be provided intravenously, subcutaneously, topically, orally, nasally, rectally, intramuscular, intracerebrally, or by inhalation. A specific dose protocol is one involving the maximal dose or dose frequency that avoids significant undesirable side effects. A total weekly dose may be at least 0.05 μg/kg body weight, at least 0.2 μg/kg, at least 0.5 μg/kg, at least 1 μg/kg, at least 10 μg/kg, at least 100 μg/kg, at least 0.2 mg/kg, at least 1.0 mg/kg, at least 2.0 mg/kg, at least 10 mg/kg, at least 15 mg/kg, at least 20 mg/kg, at least 25 mg/kg, or at least 50 mg/kg (see, e.g., Yang, et al., 2003, New Engl. J. Med. 349:427-434; Herold, et al., 2002, New Engl. J. Med. 346:1692-1698; Liu, et al., 1999, J. Neurol. Neurosurg. Psych. 67:451-456; Portielji, et al., 2003, Cancer. Immunol. Immunother. 52: 133-144). The dose may be at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg. The doses administered to a subject may number at least 1, 2, 3, 4, 5, 6, 7, 8, 9, 10, 11, or 12, or more.

For therapeutic recombinant API5 proteins of the disclosure, the dosage administered to a patient may be 0.0001 mg/kg to 100 mg/kg of the patient's body weight. The dosage may be between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight.

The dosage of the therapeutic protein of the disclosure may be calculated using the patient's weight in kilograms (kg) multiplied by the dose to be administered in mg/kg. The dosage of the proteins of the disclosure may be 150 μg/kg or less, 125 μg/kg or less, 100 μg/kg or less, 95 μg/kg or less, 90 μg/kg or less, 85 μ/kg or less, 80 μ/kg or less, 75 μ/kg or less, 70 μ/kg or less, 65 μ/kg or less, 60 μ/kg or less, 55 μ/kg or less, 50 μ/kg or less, 45 μ/kg or less, 40 μ/kg or less, 35 μ/kg or less, 30 μ/kg or less, 25 μ/kg or less, 20 μ/kg or less, 15 μ/kg or less, 10 μ/kg or less, 5 μ/kg or less, 2.5 μ/kg or less, 2 μ/kg or less, 1.5 μ/kg or less, 1 μ/kg or less, 0.5 μ/kg or less, or 0.1 μ/kg or less of a patient's body weight.

Unit dose of the therapeutic proteins of the disclosure may be 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 m g, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

The dosage of the therapeutic proteins of the disclosure may achieve a serum titer of at least 0.1 μg/ml, at least 0.5 μg/ml, at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 v, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml/ml, or at least 400 μg/ml/ml in a subject. Alternatively, the dosage of the antibodies of the disclosure may achieve a serum titer of at least 0.1 μg/ml, at least 0.5 μg/ml, at least 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml in the subject.

Doses of therapeutic proteins of the disclosure may be repeated and the administrations may be separated by at least 1 day, 2 days, 3 days, 5 days, 10 days, 15 days, 30 days, 45 days, 2 months, 75 days, 3 months, or at least 6 months.

An effective amount for a particular patient may vary depending on factors such as the condition being treated, the overall health of the patient, the method route and dose of administration and the severity of side effects (see, e.g., Maynard, et al., 1996, A Handbook of SOPs for Good Clinical Practice, Interpharm Press, Boca Raton, Fla.; Dent, 2001, Good Laboratory and Good Clinical Practice, Urch Publ, London, UK).

The route of administration may be by, e.g., topical or cutaneous application, injection or infusion by intravenous, intraperitoneal, intracerebral, intramuscular, intraocular, intraarterial, intracerebrospinal, intralesional, or by sustained release systems or an implant (see, e.g., Sidman et al., 1983, Biopolymers 22:547-556; Langer, et al., 1981, J. Biomed. Mater. Res. 15: 167-277; Langer, 1982, Chem. Tech. 12:98-105; Epstein, et al., 1985, Proc. Natl. Acad. Sci. USA 82:3688-3692; Hwang, et al., 1980, Proc. Natl. Acad. Sci. USA 77:4030-4034; U.S. Pat. Nos. 6,350,466 and 6,316,024). Where necessary, the composition may also include a solubilizing agent and a local anesthetic such as lidocaine to ease pain at the site of the injection. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety. In one embodiment, an engineered antibody or engineered antibody conjugate, combination therapy, or a composition of the disclosure is administered using Alkermes AIR™ pulmonary drug delivery technology (Alkermes, Inc., Cambridge, Mass.).

The frequency of dosing will depend upon the pharmacokinetic parameters of the recombinant API5 protein in the formulation being used. Typically, a clinician will administer the composition until a dosage is reached that achieves the desired effect. The composition can therefore be administered as a single dose, as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages can be ascertained through use of appropriate dose-response data.

The route of administration of the pharmaceutical composition is in accord with known methods, e.g., orally; through injection by subcutaneous, intravenous, intraperitoneal, intracerebral (intraparenchymal), intracerebroventricular, intramuscular, intraocular, intraarterial, intraportal, or intralesional routes; by sustained release systems (which may also be injected); or by implantation devices. Where desired, the compositions can be administered by bolus injection or continuously by infusion, or by implantation device.

Alternatively or additionally, the composition can be administered locally via implantation of a membrane, sponge, or other appropriate material onto which the desired molecule has been absorbed or encapsulated. Where an implantation device is used, the device can be implanted into any suitable tissue or organ, and delivery of the desired molecule can be via diffusion, timed-release bolus, or continuous administration. In order to deliver drug, e.g., a recombinant API5 protein as disclosed herein, at a predetermined rate such that the drug concentration can be maintained at a desired therapeutically effective level over an extended period, a variety of different approaches can be employed. In one example, a hydrogel comprising a polymer such as a gelatin (e.g., bovine gelatin, human gelatin, or gelatin from another source) or a naturally-occurring or a synthetically generated polymer can be employed. Any percentage of polymer (e.g., gelatin) can be employed in a hydrogel, such as 5, 10, 15 or 20%. The selection of an appropriate concentration can depend on a variety of factors, such as the therapeutic profile desired and the pharmacokinetic profile of the therapeutic molecule.

Examples of polymers that can be incorporated into a hydrogel include polyethylene glycol (“PEG”), polyethylene oxide, polyethylene oxide-co-polypropylene oxide, co-polyethylene oxide block or random copolymers, polyvinyl alcohol, poly(vinyl pyrrolidinone), poly(amino acids), dextran, heparin, polysaccharides, polyethers and the like.

Another factor that can be considered when generating a hydrogel formulation is the degree of crosslinking in the hydrogel and the crosslinking agent. In one embodiment, cross-linking can be achieved via a methacrylation reaction involving methacrylic anhydride. In some situations, a high degree of cross-linking may be desirable while in other situations a lower degree of crosslinking is preferred. In some cases a higher degree of crosslinking provides a longer sustained release. A higher degree of crosslinking may provide a firmer hydrogel and a longer period over which drug is delivered. Any ratio of polymer to crosslinking agent (e.g., methacrylic anhydride) can be employed to generate a hydrogel with desired properties. For example, the ratio of polymer to crosslinker can be, e.g., 8:1, 16:1, 24:1, or 32:1. For example, when the hydrogel polymer is gelatin and the crosslinker is methacrylate, ratios of 8:1, 16:1, 24:1, or 32:1 methyacrylic anhydride:gelatin can be employed.

One skilled in the art recognizes that different methods of delivery may be utilized to administer a polynucleotide (e.g., an mRNA) or vector into a cell. Examples include: (1) methods utilizing physical means, such as electroporation (electricity), a gene gun (physical force) or applying large volumes of a liquid (pressure); and (2) methods wherein the vector is complexed to another entity, such as a liposome, aggregated protein or transporter molecule.

Furthermore, the actual dose and schedule can vary depending on whether the compositions are administered in combination with other compositions, or depending on interindividual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of polynucleotide or vector to be added per cell will likely vary with the length and stability of the therapeutic gene inserted in the polynucleotide or vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

The polynucleotide molecule may also contain a suicide gene i.e., a gene which encodes a product that can be used to destroy the cell. In many gene therapy situations, it is desirable to be able to express a gene for therapeutic purposes in a host cell but also to have the capacity to destroy the host cell at will. The therapeutic agent can be linked to a suicide gene, whose expression is not activated in the absence of an activator compound. When death of the cell in which both the agent and the suicide gene have been introduced is desired, the activator compound is administered to the cell thereby activating expression of the suicide gene and killing the cell. Examples of suicide gene/prodrug combinations which may be used are herpes simplex virus-thymidine kinase (HSV-tk) and ganciclovir, acyclovir; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

### Methods of Treatment

Recombinant API5 proteins, polynucleotides, and vectors described herein and pharmaceutical compositions comprising the recombinant API5 proteins, polynucleotides, or vectors can be used to protect epithelial cells (e.g., intestinal epithelial cell such as Paneth cells) from cell death. Accordingly, the proteins, polynucleotides, and vectors of the invention can be used to restore an intestinal epithelial barrier and, thus, can be used to treat a variety of diseases or disorders that have a disrupted intestinal epithelial barrier. The disrupted intestinal epithelial barrier may be due to inflammatory disease of the gastrointestinal tract. In addition, the invention provides for use of the recombinant API5 proteins, polynucleotides, or vectors, or pharmaceutical compositions thereof, of this disclosure in the manufacture of a medicament for use in treatment or prevention of diseases or disorders that have a disrupted intestinal epithelial barrier. Examples of diseases or disorders that can be treated with the recombinant API5 proteins, polynucleotides, or vectors, or pharmaceutical compositions thereof, include, but are not limited to, inflammatory bowel disease (e.g., Crohn's disease, ulcerative colitis), graft-versus-host disease, pouchitis, immune checkpoint inhibitor associated colitis, radiation induced gastrointestinal toxicity, irritable bowel syndrome, short bowel syndrome, infectious gastroenteritis, or celiac disease.

In application, a disorder or condition can be treated by administering a recombinant API5 protein, polynucleotide, or vector, or a pharmaceutical composition thereof, as described herein, to a patient in need thereof in the amount of a therapeutically effective dose. The administration can be performed as described herein, such as by intravenous injection, intrarectal injection, intraperitoneal injection, intramuscular injection, orally in the form of a tablet or liquid formation, or delivery through endoscopy. In most situations, a desired dosage can be determined by a clinician, as described herein, and can represent a therapeutically effective dose of a recombinant API5 protein, polynucleotide, or vector. It will be apparent to those of skill in the art that a therapeutically effective dose will depend, inter alia, upon the administration schedule, the unit dose of agent administered, whether the composition is administered in combination with other therapeutic agents, and the health of the recipient. The term “therapeutically effective dose,” as used herein, means that amount of recombinant API5 protein, polynucleotide, or vector, that elicits the biological or medicinal response in a tissue system, animal, or human being sought by a researcher, medical doctor, or other clinician, which includes alleviation of the symptoms of the disease or disorder being treated.

In some embodiments, the recombinant API5 protein, polynucleotide, or vector, or a pharmaceutical composition thereof, is administered in combination with one or more additional agents. In some embodiments, the additional agent is an agent that inhibits TNFα and/or lymphocyte migration. Exemplary agents suitable for use in the methods of the present disclosure include, but are not limited to, integrin inhibitors (e.g., vedolizumab (Entyvio), etrolizumab, PN-943, ZP10000, or MORF-057), or sphingosine-1-phosphate (S1P) receptor modulators (e.g., fingolimod, ozanimod, etrasimod, or amiselimod).

### EXAMPLES

The following examples are provided to further describe some of the embodiments disclosed herein. The examples are intended to illustrate, not to limit, the disclosed embodiments.

### Example 1. γδ T Cells Protect Paneth Cells and Intestinal Organoids from Cell Death

Paneth cells are secretory IECs of the small intestine that protect the epithelial stem cell niche through the production of antimicrobials and growth factors. Both mice and humans harboring the common T300A variant of ATG16L1 display loss of Paneth cells^{1,2}. Subsequent studies confirmed the essential role of Paneth cells in the intestinal barrier, and provided mechanistic insight into the biology of these critical cells³⁻⁷.

In a virus-triggered animal model of Crohn's disease, ATG16L1 prevents Paneth cells from undergoing TNFα-induced necroptosis, a form of programmed necrosis³. Mechanistic experiments with Atg16L1^{-/-} enteroids from mice indicate that IEC necroptosis occurs downstream of a defect in organelle homeostasis, a known function of ATG16L1 in the cellular process of autophagy. This cellular stress response leads to aberrant JAK/STAT and RIPK signaling in response to TNFα, and that blocking JAK/STAT or RIPK, or TNFα restores Paneth cells and reverses intestinal disease in virally-infected Atg16L1 mutant mice. Additionally, enteroids generated from Crohn's disease patients homozygous for ATG16L1^{T300A} were susceptible to TNFα-induced cell death, and viability was restored by chemical inhibitors of JAK/STAT and RIPK signaling⁸. Therefore, ATG16L1^{T300A} confers susceptibility to cell death in human IECs in a manner similar to mice.

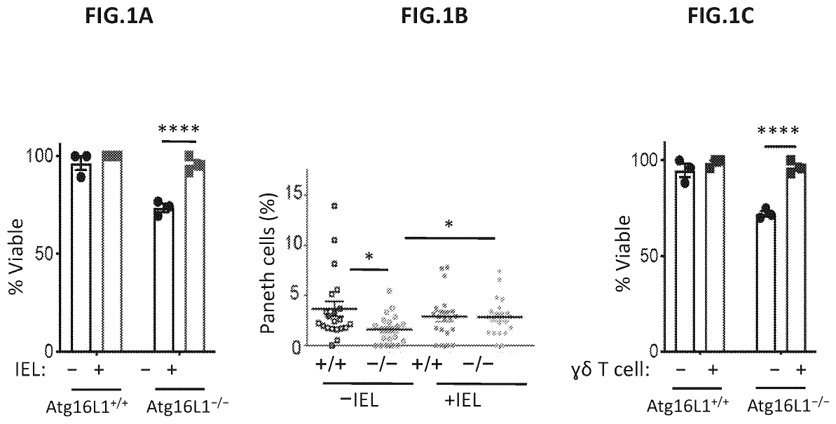

3D intestinal epithelial organoids generated from Atg16L1^{-/-} mice (villin-Cre Atg16L1^{flox/flox} mice) recreate the loss of Paneth cells, a hallmark of Crohn's disease. Necrotic cell death of Paneth cells then leads to loss of viability of the organoid. FIGS. 1A-1C show that γδ T cells protect Paneth cells and intestinal organoids from cell death. Addition of intra-epithelial lymphocytes (IELs) to Atg16L1^{-/-} organoids restores viability (FIG. 1A) and the proportion (FIG. 1B) of Paneth cells to similar levels as control Atg16L1^{+/+} wild-type organoids. IELs include a heterogeneous group of immune cell types. Among the different IEL subtypes, γδ T cells were the ones that mediate protection ofAtg16L1^{-/-} organoids (FIG. 1C).

### Example 2. Inhibition of the Protective Function of γδ T Cells is Associated with Paneth Cell Defects

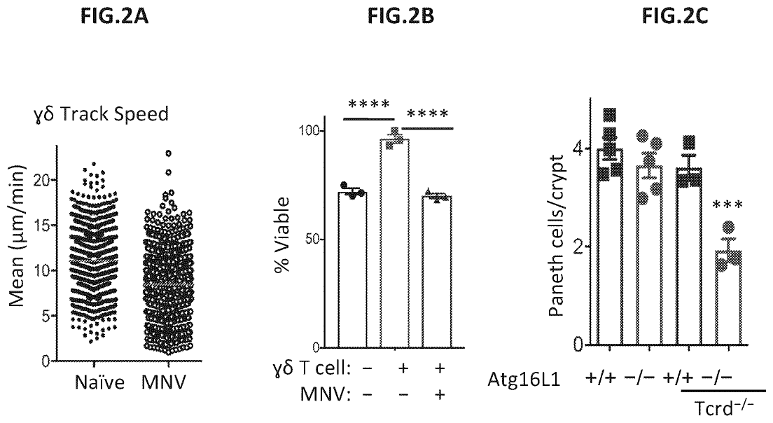

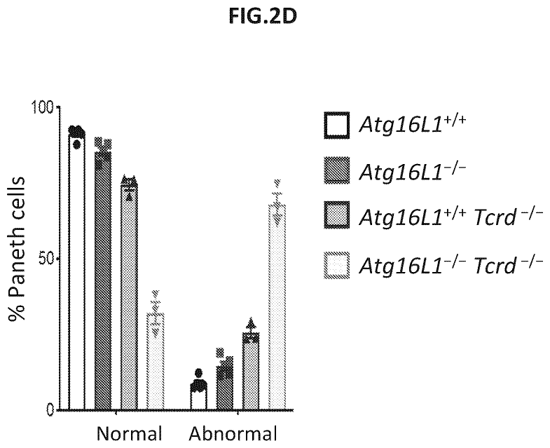

In the preclinical animal model of Crohn's disease, murine norovirus (MNV) infection triggers loss of Paneth cells and downstream intestinal disease in Atg16L1^{-/-} mice. MNV inhibits γδ T cell mobility, a sign that their activity is altered (FIG. 2A). It was found that γδ T cells from uninfected mice, but not MNV-infected mice, promote Atg16L1^{-/-} organoid viability, indicating the virus interferes with the protective effect of these cells (FIG. 2B). If MNV triggers loss of Paneth cells in Atg16L1^{-/-} mice by interfering with γδ T cells, then removing γδ T cells from these mice should mimic this effect of the virus. Indeed, double knockout mice generated by crossing Atg16L1^{-/-} mice with mice that lack γδ T cells (Tcrd^{-/-}) displayed a loss of Paneth cells. These results were supported by lysozyme immunofluorescence microscopy, which indicated that the remaining Paneth cells displayed abnormal staining patterns of this antimicrobial molecule in Atg16L1^{-/-} Tcrd^{-/-} mice compared to single knockout controls (FIG. 2D). These data demonstrated that inhibition of the protective function of γδ T cells is associated with Paneth cell defects.

### Example 3. Identification of API5 as a Secreted Molecule from γδ T Cells

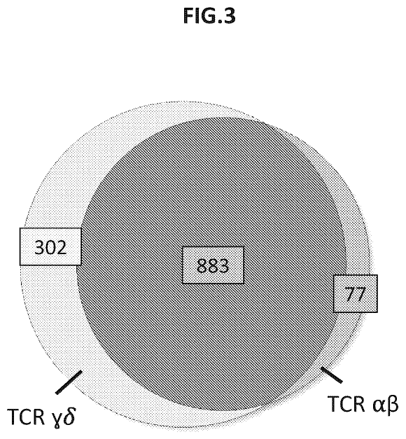

Supernatant from FACS-sorted TCRγδ⁺ cells (γδ T cells) and TCRαβ⁺ cells (conventional T cells) were analyzed by mass spectrometry. A total of 1200 proteins were identified. The numbers of overlapping and distinct proteins in the two samples are shown in FIG. 3A. STRING pathway analysis showed enrichment of extracellular proteins (Table 3), supporting the validity of the approach.

TABLE 3
STRING pathway analysis

| | | | |
|---|---|---|---|
| | | | false |
| pathway | | count in | discovery |
| ID | pathway description | gene set | rate |

Biological Process (GO)

| | | | |
|---|---|---|---|
| GO:0008152 | metabolic process | 568 | 2.75e−57 |
| GO:0071704 | organic substance | 531 | 2.75e−57 |
| | metabolic process | | |
| GO:0044237 | cellular metabolic process | 506 | 2.76e−54 |
| GO:0044238 | primary metabolic process | 512 | 3.59e−53 |
| GO:1901564 | organonitrogen compound | 181 | 8.17e−51 |
| | metabolic process | | |
| | | | (more . . . ) |

Molecular Function (GO)

| | | | |
|---|---|---|---|
| GO:0003723 | RNA binding | 258 | 2.72e−95 |
| GO.0044822 | poly(A) RNA binding | 224 | 1.31e−92 |
| GO.0005488 | binding | 638 | 3.23e−67 |
| GO.0003824 | catalytic activity | 434 | 1.04e−62 |
| GO:0003723 | heterocyclic | 403 | 3.46e−52 |
| | compound binding | | |
| | | | (more . . . ) |

Cellular Component (GO)

| | | | |
|---|---|---|---|
| GO.0065010 | extracellular membrane- | 503 | 2.55e−235 |
| | bounded organelle | | |
| GO.0070062 | extracellular exosome | 502 | 6.03e−235 |
| GO.0031988 | membrane-bounded | 518 | 1.11e−203 |
| | vesicle | | |
| GO.0031982 | vesicle | 522 | 8.11e−200 |
| GO.0044421 | extracellular | 521 | 1.6e−190 |
| | region part | | |
| | | | (more . . . ) |

Table 4 lists the proteins with the highest peptide spectral matches (PSMs) unique to TCRγδ⁺ cell supernatant. API5 was among the top hits of the 302 proteins and was chosen for further analyses.

TABLE 4
Proteins unique to TCRγδ⁺ cell supernatant

| Protein | Gene | # PSM |
|---|---|---|
| Ornithine aminotransferase, mitochondrial | Oat | 18 |
| Fructose-bisphosphate aldolase C | Aldoc | 16 |
| Integrin alpha-E | Itgae | 12 |
| Peptidyl-prolyl cis-trans isomerase F, mitochondrial | Pp [symbol] | 11 |
| Keratin, type II cytoskeletal 8 | Krt8 | 10 |
| C-C motif chemokine 4 | Ccl4 | 8 |
| Kinesin- [symbol] heavy chain | K [symbol] b | 8 |
| Lupus [symbol] protein homolog | [symbol] b | 8 |
| Hydroxyacylglutathione hydrolase, mitochondrial (Fragment) | Hagh | 8 |
| Eukaryotic translation initiation factor 5A (Fragment) | E [symbol] 5a | 8 |
| Apoptosis-associated speck-like protein containing a CARD | Pycard | 7 |
| Calcium-activated chloride channel regulator 1 | Cka1 | 7 |
| Fructose-1,8-bisphosphatase isozyme 2 | Pep2 | 7 |
| Host cell factor 1 | Hcfc1 | 7 |
| Multidrug resistance protein 1A | Abcb1a | 7 |
| Proteasome subunit beta type-5 | Psmb5 | 7 |
| Apoptosis inhibitor 5 | Ap [symbol] 5 | 6 |
| Catheosin 5 | Ctss | 6 |
| Succinate dehydrogenase (ubiquinone) flavoprotein subunit, mitochondrial | Sdha | 6 |
| Isoform 8 of Heterochromatin protein 1-binding protein [symbol] | Hpthp [symbol] | 6 |
| Pyrethroid hydrolase Ces2e | Ces2e | 6 |
| Heparan N-sulfatase | [symbol] gsh | 6 |
| Acylcarnitine hydrolase | Ces2c | 6 |
| A [symbol] met protein | Manf | 6 |
| Density-regulated protein (fragment) | Danr | 6 |
| Thioredoxin domain-containing protein 5 | Txndc5 | 6 |
| ANKAS_HUMAN Annexin AS | ANKAS | 5 |
| Ribosomal protein | Rpl10a | 5 |
| Anterior gradient protein 2 homolog | Agr2 | 5 |
| Isoform 2 of protein archease | Zbtb8os | 5 |
| CD48 antigen | Cd48 | 5 |
| Coatomer subunit epsilon | Co [symbol] | 5 |
| Non-P [symbol] domain-containing octamer-binding protein | Nono | 5 |
| AOS ribosomal protein S17 | Rps17 | 5 |
| Small nuclear ribonucleoprotein-associated protein 8 | Snrpb | 5 |
| Splicing factor, proline- and glutamate-rich | Sfpg | 5 |
| Staphlococcal nuclease domain-containing protein 1 | Snd1 | 5 |
| Phenylalanine- [symbol] NA ligase beta subunit | Farsb | 5 |

[symbol]

indicates data missing or illegible when filed

### Example 4. API5 Protects Intestinal Organoids and Restores Paneth Cells

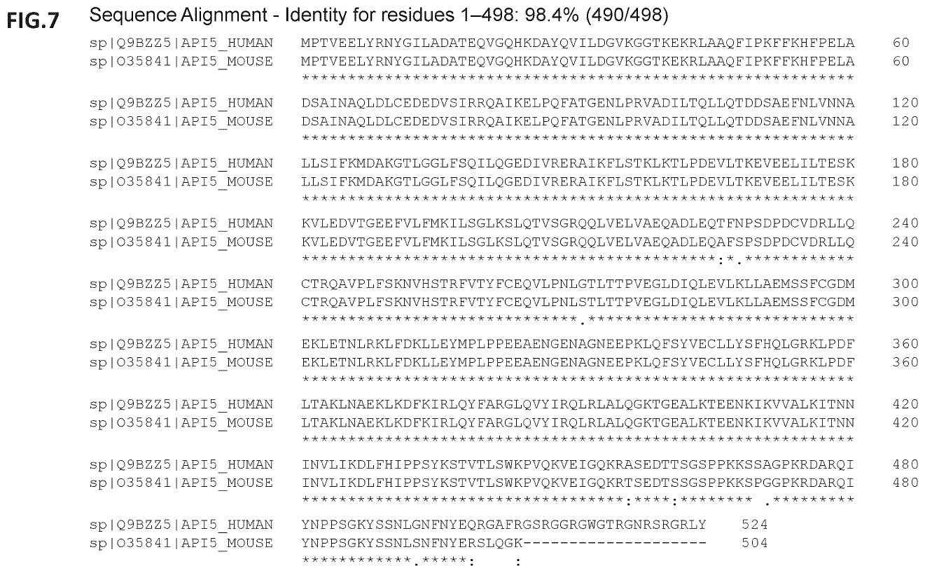

Recombinant human API5 (rhAPI5) was generated using residues 1-448 of the wild-type human API5 protein. The sequence identity of this fragment between human and mouse is 99.3% (445/448) (see FIG. 7). This fragment was fused to an N-terminal tag encoding His₆ (SEQ ID NO: 78), Avi-tag and the cleavage site for the TEV protease. The rhAPI5 construct is provided below.

WT API5 (residues 1-448 of human API5)-the N-terminal tag encoding
His₆ (SEQ ID NO: 78), Avi-tag and the cleavage site for the TEV protease
is underlined
(SEQ ID NO: 3)
MKHHHHHHSSGLNDIFEAQKIEWHEENLYFQGS
MPTVEELYRNYGILADATEQVGQHKDAYQVILDGVKGGTKEKRLAAQFIPKFFKHFPELA
DSAINAQLDLCEDEDVSIRRQAIKELPQFATGENLPRVADILTQLLQTDDSAEFNLVNNA
LLSIFKMDAKGTLGGLESQILQGEDIVRERAIKFLSTKLKTLPDEVLTKEVEELILTESK
KVLEDVTGEEFVLFMKILSGLKSLQTVSGRQQLVELVAEQADLEQTFNPSDPDCVDRLLQ
CTRQAVPLESKNVHSTRFVTYFCEQVLPNLGTLTTPVEGLDIQLEVLKLLAEMSSFCGDM
EKLETNLRKLFDKLLEYMPLPPEEAENGENAGNEEPKLQFSYVECLLYSFHQLGRKLPDF
LTAKLNAEKLKDFKIRLQYFARGLQVYIRQLRLALQGKTGEALKTEENKIKVVALKITNN
INVLIKDLFHIPPSYKSTVTLSWKPVQK

His-tagged rhAPI5 was purified from E. coli expressing the construct with a Ni-Sepharose column using a standard procedure that also includes a washing step to reduce endotoxins. The protein was further purified using size-exclusion chromatography.

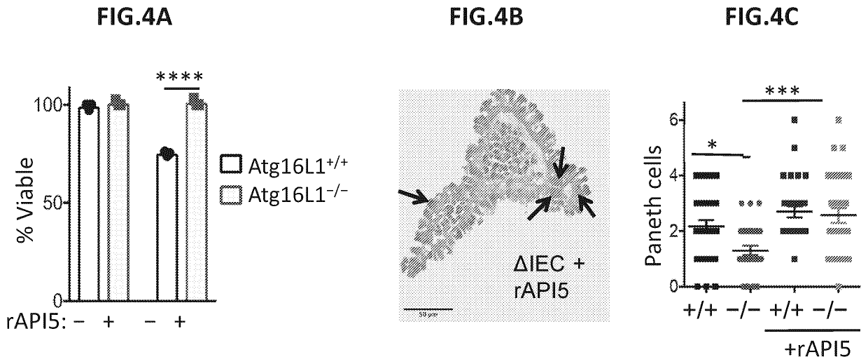

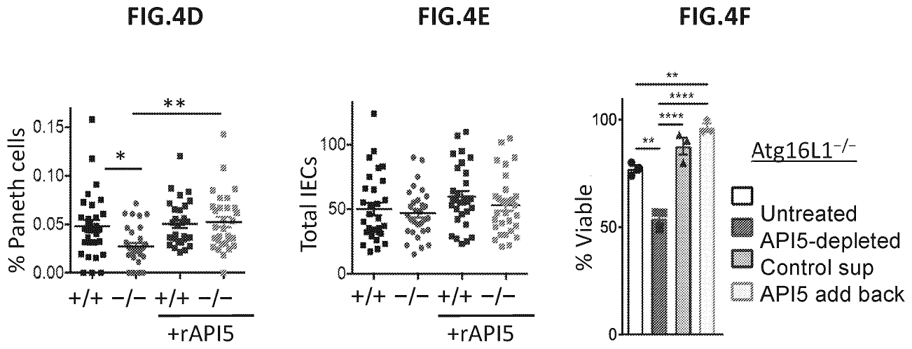

As shown in FIG. 4A, 50 nM recombinant human API5 (rhAPI5) restores Atg16L1^{-/-} organoid viability. H&E-staining (FIG. 4B) demonstrated that rhAPI5 restores Paneth cells. Quantification of absolute Paneth cell numbers per organoid (FIG. 4C) and percent of total intestinal epithelial cells (IECs) (FIG. 4D) confirmed a restoration of Paneth cells. Total IECs did not increase (FIG. 4E) indicating that the effect of rAPI5 is Paneth cell-specific and not as a non-specific growth factor. As indicated in FIG. 1A, IELs protect Atg16L1^{-/-} organoids. Adding IEL supernatant in which API5 is depleted with an antibody exacerbates Atg16L1^{-/-} organoid death, while adding rAPI5 to the depleted supernatant results in similar protection as the intact IEL supernatant (Control sup) (FIG. 4F).

### Example 5. Binding Interface Residues of API5 are Necessary for Protective Effects

Mutations were introduced to API5 to test the role of surface resides predicted to mediate protein interactions based on the available crystal structure. Mutant 1 encodes API5 with Y8K;Y11K amino acid changes targeting hydrophobic residues on a concave surface. Mutant 2 encodes API5 with E184K;D185K amino acid changes that change the surface charge. Mutant 3 combines all four amino acid changes. The mutant constructs are provided below. Mutant rhAPI5 proteins were purified similarly as described in Example 4.

API5 (Y8K; Y11K)-mutations in bold; the N-terminal tag encoding His₆
(SEQ ID NO: 78), Avi-tag and the cleavage site for the TEV protease is
underlined
(SEQ ID NO: 4)
MKHHHHHHSSGLNDIFEAQKIEWHEENLYFQGS
MPTVEELKRNKGILADATEQVGQHKDAYQVILDGVKGGTKEKRLAAQFIPKFFKHFPELA
DSAINAQLDLCEDEDVSIRRQAIKELPQFATGENLPRVADILTQLLQTDDSAEFNLVNNA
LLSIFKMDAKGTLGGLESQILQGEDIVRERAIKFLSTKLKTLPDEVLTKEVEELILTESK
KVLEDVTGEEFVLFMKILSGLKSLQTVSGRQQLVELVAEQADLEQTENPSDPDCVDRLLQ
CTRQAVPLFSKNVHSTRFVTYFCEQVLPNLGTLTTPVEGLDIQLEVLKLLAEMSSFCGDM
EKLETNLRKLFDKLLEYMPLPPEEAENGENAGNEEPKLQFSYVECLLYSFHQLGRKLPDF
LTAKLNAEKLKDFKIRLQYFARGLQVYIRQLRLALQGKTGEALKTEENKIKVVALKITNN
INVLIKDLFHIPPSYKSTVTLSWKPVQK
API5 (E184K; D185K)-mutations in bold; the N-terminal tag encoding His₆
(SEQ ID NO: 78), Avi-tag and the cleavage site for the TEV protease is
underlined
(SEQ ID NO: 5)
MKHHHHHHSSGLNDIFEAQKIEWHEENLYFQGS
MPTVEELYRNYGILADATEQVGQHKDAYQVILDGVKGGTKEKRLAAQFIPKFFKHFPELA
DSAINAQLDLCEDEDVSIRRQAIKELPQFATGENLPRVADILTQLLQTDDSAEFNLVNNA
LLSIFKMDAKGTLGGLFSQILQGEDIVRERAIKFLSTKLKTLPDEVLTKEVEELILTESK
KVLKKVTGEEFVLFMKILSGLKSLQTVSGRQQLVELVAEQADLEQTENPSDPDCVDRLLQ
CTRQAVPLFSKNVHSTRFVTYFCEQVLPNLGTLTTPVEGLDIQLEVLKLLAEMSSFCGDM
EKLETNLRKLFDKLLEYMPLPPEEAENGENAGNEEPKLQFSYVECLLYSFHQLGRKLPDF
LTAKLNAEKLKDFKIRLQYFARGLQVYIRQLRLALQGKTGEALKTEENKIKVVALKITNN
INVLIKDLFHIPPSYKSTVTLSWKPVQK
API5 (Y8K; Y11K; E184K; D185K)-mutations in bold; the N-terminal tag
encoding His₆ (SEQ ID NO: 78), Avi-tag and the cleavage site for the TEV
protease is underlined
(SEQ ID NO: 11)
MKHHHHHHSSGLNDIFEAQKIEWHEENLYFQGS
MPTVEELKRNKGILADATEQVGQHKDAYQVILDGVKGGTKEKRLAAQFIPKFFKHFPELA
DSAINAQLDLCEDEDVSIRRQAIKELPQFATGENLPRVADILTQLLQTDDSAEFNLVNNA
LLSIFKMDAKGTLGGLESQILQGEDIVRERAIKFLSTKLKTLPDEVLTKEVEELILTESK
KVLKKVTGEEFVLFMKILSGLKSLQTVSGRQQLVELVAEQADLEQTENPSDPDCVDRLLQ
CTRQAVPLESKNVHSTRFVTYFCEQVLPNLGTLTTPVEGLDIQLEVLKLLAEMSSFCGDM
EKLETNLRKLFDKLLEYMPLPPEEAENGENAGNEEPKLQFSYVECLLYSFHQLGRKLPDF
LTAKLNAEKLKDFKIRLQYFARGLQVYIRQLRLALQGKTGEALKTEENKIKVVALKITNN
INVLIKDLFHIPPSYKSTVTLSWKPVQK

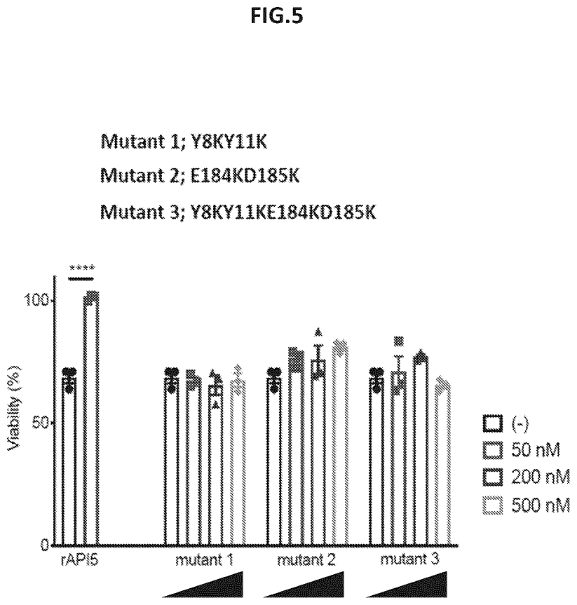

As shown in FIG. 5, first two bar graphs are controls showing that 50 nM recombinant human wild-type API5 (rhAPI5) restores Atg16L1^{-/-} organoid viability as previously indicated. The rhAPI5 variants abrogated the protective activity, even when adding excess protein up to 500 nM. These results support the specificity of the protective effect of API5.

### Example 6. API5 Prevents TNFα-Induced Loss of Epithelial Viability

TNFα blockade is a major therapy for Crohn's disease, which also ameliorates disease in the preclinical Atg16L1 mutant animal model. Atg16L1^{-/-} organoids undergo exacerbated necrotic cell death in the presence of 20 ng/ml TNFα due to their loss of Paneth cells, but control organoids are resistant. Administering 50 nM rhAPI5 prevents the toxic effect of TNFα to improve the viability of Atg16L1^{-/-} organoids (FIG. 6).

### Example 7. API5 Prevents Paneth Cell Loss and Protects Against Intestinal Injury in Atg-16L1 Mutant Mice

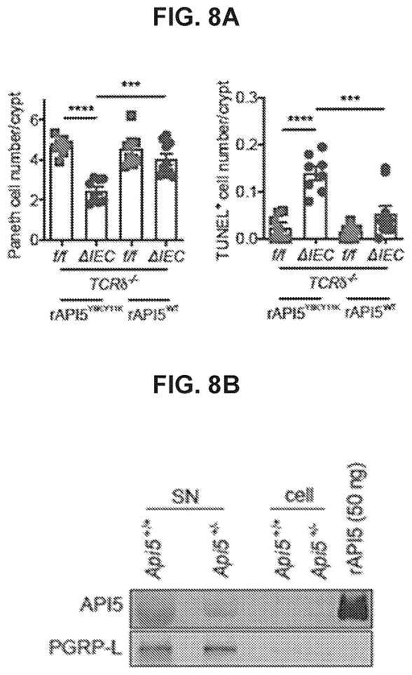

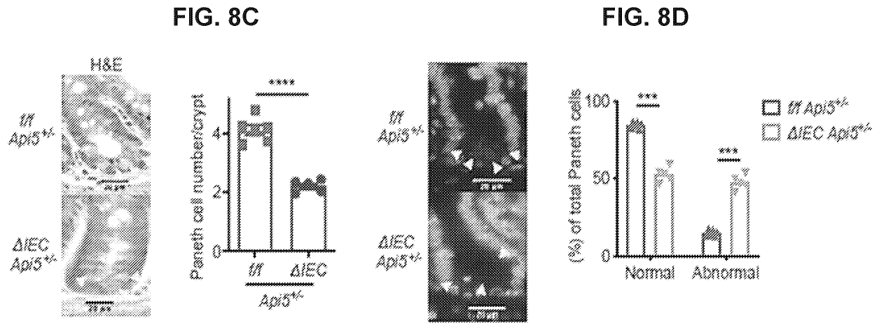

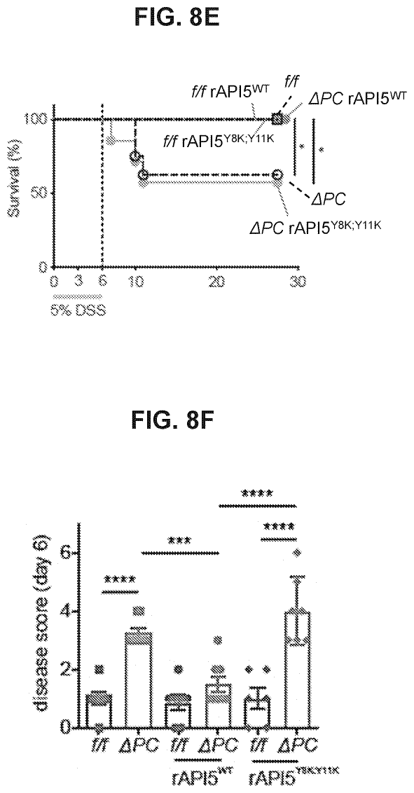

Based on the findings that API5 prevents cell death of organoids and restores Paneth cells in vitro, in vivo studies were carried out to examine whether API5 has similar protective functions in animal models. When rAPI5 was injected into Atg16L1 mutant mice deficient in γδ T cells (Atg16L1^{ΔIEC} TCRδ^{-/-}), which lack the T cells that secrete API5, it was found that the Paneth cells were restored and cell death was reduced (FIG. 8A). Atg16L1 mutant mice were then generated with partial API5 deficiency (Atg16L1^{ΔIEC} Api5^{+/-}). The partial deficiency leads to reduced secretion of API5 by γδ T cells as confirmed by Western blot analysis (FIG. 8B). The mutant mice display reduction in Paneth cells (FIG. 8C), and the remaining Paneth cells are morphologically abnormal (FIG. 8D). These results showed that API5 is necessary for preventing Paneth cell abnormalities in vivo and that the amount of API5 plays a role. Finally, the therapeutic potential of API5 was tested in an independent preclinical model. The Atg16L1^{ΔPC} mouse line was chosen in which Atg16L1 is deleted from Paneth cells (defensin-Cre Atg16L1^{f/f}) because these mice do not have deletions in API5 or γδ T cells, allowing for investigation of whether providing additional API5 is therapeutic. Administration of rAPI5 led to a complete reversal of disease (FIGS. 8E and 8F). Because the genetic lesion is restricted to Paneth cells, these results provided additional evidence of the specificity of rAPI5 treatment for these critical epithelial cells. Together, these results using three different mutant mouse lines support targeting of API5 for therapy.

### REFERENCES

1. Cadwell, K., et al. Nature, 259 (2008).
2. Cadwell, K., et al. Cell 1135 (2010).
3. Matsuzawa-Ishimoto, Y., et al. J Exp Med, 3687 (2017).
4. Adolph, T. E., et al. Nature, 272 (2013).
5. Bel, S., et al. Science 1047 (2017).
6. Lassen, K. G., et al. PNAS 7741 (2014).
7. VanDussen, K. L., et al. Gastroenterology 200 (2014).
8. Matsuzawa-Ishimoto, Y., et al. Blood 2388 (2020).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A recombinant protein comprising an apoptosis inhibitor 5 (API5) protein, or a fragment or a variant thereof.

2. The recombinant protein of claim 1, wherein the API5 protein comprises the amino acid sequence of SEQ ID NO: 1 or 2, or a sequence having at least 90% identity thereto.

3. The recombinant protein of claim 1, wherein the fragment of API5 comprises the N-terminal HEAT repeat region of API5.

4. The recombinant protein of claim 1 or 3, wherein the fragment of API5 comprises residues 1-448 of SEQ ID NO: 1.

5. The recombinant protein of claim 1 or 3, wherein the fragment of API5 comprises residues 1-206 of SEQ ID NO: 1.

6. The recombinant protein of any one of claims 1-5, wherein the API5 protein, or a fragment or a variant thereof, is genetically fused to and/or chemically conjugated to one or more heterologous moieties.

7. The recombinant protein of claim 6, wherein the one or more heterologous moieties comprise one or more affinity tags.

8. The recombinant protein of claim 7, wherein the affinity tag is a His tag, an Avi-tag, a hemagglutinin (HA) tag, a FLAG tag, a Myc tag, a GST tag, a MBP tag, a chitin binding protein tag, a calmodulin tag, a V5 tag, a streptavidin binding tag, a green fluorescent protein (GFP), YFP, RFP, CFP, mCherry, tdTomato, SUMO tag, Ubiquitin tag, or a combination thereof.

9. The recombinant protein of any one of claims 6-8, wherein the one or more heterologous moieties comprise a His₆ tag (SEQ ID NO: 78) and an Avi-tag, and optionally comprise the amino acid sequence of MKHHHHHHSSGLNDIFEAQKIEWHE (SEQ ID NO: 9).

10. The recombinant protein of any one of claims 6-9, wherein the recombinant protein further comprises a protease cleavage site between the API5 protein, or a fragment or a variant thereof, and the one or more affinity tags.

11. The recombinant protein of claim 10, wherein the protease cleavage site is a cleavage site for the TEV protease and optionally comprises the amino acid sequence of ENLYFQGS (SEQ ID NO: 10).

12. The recombinant protein of any one of claims 1, 4, and 6-11, wherein the recombinant protein comprises the amino acid sequence of SEQ ID NO: 3.

13. The recombinant protein of claims 1, 5, and 6-11, wherein the recombinant protein comprises the amino acid sequence of SEQ ID NO: 6.

14. The recombinant protein of any one of claims 6-13, wherein the one or more heterologous moieties comprise a moiety that specifically binds albumin.

15. The recombinant protein of claim 14, wherein the moiety that specifically binds albumin comprises the amino acid sequence of any one of SEQ ID NOs: 12-66, 76 and 77.

16. The recombinant protein of claim 14, wherein the moiety that specifically binds albumin is selected from Naphthalene acylsulfonamide, Diphenylcyclohexanol phosphate ester, 9-fluorenylmethoxycarbonyl (Fmoc), Fmoc derivative linked to a 16-sulfanylhexadecanoic acid through a maleimide group, Dicoumarol derivative with maleimide, Evans blue derivative with maleimide, Diflunisal-γGlu-Lys(±O2O c)-indomethacin, lithocholic acid coupled to a γGlu linker, 6-(4-(p-Iodophenyl) butanamido) hexanoate, A083/B134, A099/B344, 89D03 (Ac-WWEQDRDWDFDVFGGGTP-NH₂, SEQ ID NO: 67), acylated heptapeptide F-tag (fluorescein-EYEK(palmitate)EYE-NH₂, SEQ ID NO: 68), disulfide cyclized peptide SA21 (Ac-RLIEDICLPRWGCLWEDD-NH₂, SEQ ID NO: 69), head-to-tail cyclized peptide HSA-1 (AK*K*PGK*AK*PG with variable lysine (K*), SEQ ID NO: 70), ABD035, ABDCon, DARPins, AlbudAbs, dsFv CA645, Nanobody Nb.b201, and VNAR E06.

17. The recombinant protein of any one of claims 14-16, wherein the albumin is rat albumin, rabbit albumin, or human albumin.

18. The recombinant protein of any one of claims 14-17, wherein the moiety that specifically binds albumin is genetically fused or chemically conjugated to the N-terminus of the API5 protein, or a fragment or a variant thereof.

19. The recombinant protein of any one of claims 14-17, wherein the moiety that specifically binds albumin is genetically fused or chemically conjugated to the C-terminus of the API5 protein, or a fragment or a variant thereof.

20. The recombinant protein of any one of claims 14-19, wherein the moiety that specifically binds albumin is genetically fused or chemically conjugated to the API5 protein, or a fragment or a variant thereof via a linker.

21. The recombinant protein of any one of claims 6-20, the one or more heterologous moieties comprise a human IgG Fc domain.

22. The recombinant protein of claim 21, the Fc domain is modified to alter effector function of the domain.

23. The recombinant protein of claim 21 or 22, the Fc domain is modified to enhance the half-life of the recombinant protein.

24. The recombinant protein of any one of claims 6-23, wherein the one or more heterologous moieties comprise an albumin.

25. The recombinant protein of any one of claims 6-24, wherein the one or more heterologous moieties comprise a polyethylene glycol (PEG) polymer.

26. The recombinant protein of any one of claims 1-25, wherein the recombinant protein is modified to introduce one or more glycosylation sites in the recombinant protein.

27. An isolated polynucleotide encoding the recombinant protein of any one of claims 1-26.

28. The isolated polynucleotide of claim 27, wherein the isolated polynucleotide is an mRNA.

29. A vector comprising the polynucleotide of claim 27.

30. A host cell comprising the polynucleotide of claim 27 or the vector of claim 29.

31. A pharmaceutical composition comprising the recombinant protein of any one of claims 1-26, the polynucleotide of claims 27 or 28, or the vector of claim 29, and a pharmaceutically acceptable carrier or excipient.

32. A method of producing a recombinant protein of any one of claims 1-26, comprising growing the host cell of claim 30 under conditions where the protein encoded by the polynucleotide is expressed.

33. The method of claim 32, further comprising isolating the protein.

34. A method of protecting an epithelial cell from cell death, comprising contacting the epithelial cell with a therapeutically effective amount of the recombinant protein of any one of claims 1-26, the polynucleotide of claim 27 or 28, the vector of claim 29, or the pharmaceutical composition of claim 31.

35. The method of claim 34, wherein the epithelial cell is an intestinal epithelial cell.

36. The method of claim 35, wherein the epithelial cell is a Paneth cell.

37. A method of restoring an intestinal epithelial barrier in a subject in need thereof, comprising administering to the subject a therapeutically effective amount of the recombinant protein of any one of claims 1-26, the polynucleotide of claim 27 or 28, the vector of claim 29, or the pharmaceutical composition of claim 31.

38. The method of claim 37, wherein the subject has a gastrointestinal disease.

39. The method of claim 37, wherein the gastrointestinal disease is an inflammatory bowel disease, graft-versus-host disease, pouchitis, immune checkpoint inhibitor associated colitis, radiation induced gastrointestinal toxicity, irritable bowel syndrome, short bowel syndrome, infectious gastroenteritis, or celiac disease.

40. The method of claim 39, wherein the inflammatory bowel disease is Crohn's disease.

41. The method of claim 39, wherein the inflammatory bowel disease is ulcerative colitis.

42. A method of treating a gastrointestinal disease in a subject in need thereof, comprising administering to the subject a therapeutically effective amount of the recombinant protein of any one of claims 1-26, the polynucleotide of claim 27 or 28, the vector of claim 29, or the pharmaceutical composition of claim 31.

43. The method of claim 42, wherein the gastrointestinal disease is inflammatory bowel disease, graft-versus-host disease, pouchitis, immune checkpoint inhibitor associated colitis, radiation induced gastrointestinal toxicity, irritable bowel syndrome, short bowel syndrome, infectious gastroenteritis, or celiac disease.

44. The method of claim 43, wherein the inflammatory bowel disease is Crohn's disease.

45. The method of claim 43, wherein the inflammatory bowel disease is ulcerative colitis.

46. The method of any one of claims 37-44, wherein the recombinant protein, the polynucleotide, the vector, or the pharmaceutical composition is administered intravenously, orally, intrarectally, or via delivery through endoscopy.

47. The method of any one of claims 37-46, further comprising administering one or more additional agents.

48. The method of claim 47, wherein the one or more additional agents inhibit TNFα and/or lymphocyte migration.

49. The method of claim 48, wherein the one or more additional agents comprise an integrin inhibitor or a sphingosine-1-phosphate (S1P) receptor modulator.

50. The method of claim 49, wherein the integrin inhibitor is vedolizumab (Entyvio), etrolizumab, PN-943, ZP10000, or MORF-057.

51. The method of claim 49, wherein the S1P receptor modulator is fingolimod, ozanimod, etrasimod, or amiselimod.

52. The method of any one of claims 37-51, wherein the subject is human.
